SEQUENCE LISTING (55 sequences)

SEQ ID NO: 1 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagaggcctggacaaggccttgagtggatcggagagattgatccttctgatagttatactaactacaatcaaaagttcaagggcaaggccaagttgactgtagacaaatcctccagcacagcctacatggaagtcagcagcctgacatctgaggactctgcggtctattactgtgcaagaggtagcaacccttactactatgctatggactactggggtcaaggaacctcagtcaccgtctcctca

SEQ ID NO: 2 - DNA, 360 bp, synthetic construct
  source 1..360: mol_type = other DNA
caggtgcaactgcagcagcctggggctgaagtggtgaagcctggggcttcagtgagactgtcctgcgagacttctggctacaccttcaccagcaactggatgcactgggtgaagcagaggcctgaccaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 3 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagagacctggacaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 4 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgaagcagaggcctggacaaggacttgagtggatcggagagattgatccttctgatagttatactaactacaatcaaaagttcaagggcaaggccaagttgactgtagacaaatcctccagcacagcctacatggaagtcagcgacctgacagctgaggactctgcggtctattactgtgcaagaggtagcaacccttactactatgctatggactactggggtcaaggaacctcagtcaccgtctcctca

SEQ ID NO: 5 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagacgcctggaaaaggacttgagtggatcggagagattgatccttctgatagttatactaactacaatcaaaagttcgatggcaaggccaagttgactgtagacaaatcctccagcacagcctacatggaagtcagcgacctgacagctgaggactctgcgacctattactgtgcaagaggtagcaacccttactactatgctatggactactggggtcaaggaacctcagtcaccgtctcctca

SEQ ID NO: 6 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgagacaggcacctggaaaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 7 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagaggcctggacaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 8 - DNA, 311 bp, synthetic construct
  source 1..311: mol_type = other DNA
caaattgttctcacccagtctccagcaatcatgtctgcatctccaggggagaaggtcaccatgacctgcagtgccagctcaggtgtcaactacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 9 - DNA, 311 bp, synthetic construct
  source 1..311: mol_type = other DNA
caaattgctctcacccaggagccagcaatcatgtctgcatctccaggggagaaggtcaccatgacctgcagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 10 - DNA, 311 bp, synthetic construct
  source 1..311: mol_type = other DNA
gacattgttctcacccagtctccagcaactttgtctgcatctccaggggagaaggtcaccatgacctgtagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 11 - DNA, 311 bp, synthetic construct
  source 1..311: mol_type = other DNA
caaattgttctcacccagtctccagcaactttgtctgcatctccaggggagaaggctaccatgacctgcagtgccagctcaggtgctaactacatgcactggtaccagcagaagccaggcacctcccccaaaagatggatttatgacacatccaaactggcttctggagtccctgctcgcttcagtggcagtgggtctgggacctcttattctctcacaatcgagagcatggaggctgaagatgctgccacttattactgccatcagcgaggtagttacacgttcggaggggggaccaagctggaaataaa

SEQ ID NO: 12 - DNA, 310 bp, synthetic construct
  source 1..310: mol_type = other DNA
caaattgttctcacccagtctccagcaactttgtctgcatctccaggggagaaggctacatgacctgtagtgccagctcaggtgtcaactacatgcactggtaccagcagaagccaggcacctcccccaaaagatggatttatgacacagacaaaacggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 13 - protein, 120 aa, synthetic construct
QVQLQQPGAEVVKPGASVRLSCKTSGYTFTSNWMHWVKQRPGQGLEWIGEIDPSDSYTNYNQKFKGKAKLTVDKSSSTAYMEVSSLTSEDSAVYYCARGSNPYYYAMDYWGQGTSVTVSS

SEQ ID NO: 14 - protein, 120 aa, synthetic construct
QVQLQQPGAEVVKPGASVRLSCETSGYTFTSNWMHWVKQRPDQGLEWIGEIDPSDSYTNYNQKFKGKAELTVDKSSSTAYMEVSDLTSEDSAVYYCARGSNPYYYAMDYWGQGTSVTVSS

SEQ ID NO: 15 - protein, 120 aa, synthetic construct
QVQLQQPGAEVVKPGASVRLSCKTSGYTFTSNWMHWVKQRPGQGLEWIGEIDPSDSYTNYNQKFKGKAELTVDKSSSTAYMEVSDLTSEDSAVYYCARGSNPYYYAMDYWGQGTSVTVSS

SEQ ID NO: 16 - protein, 120 aa, synthetic construct
QVQLEQPGAEVKKPGASVKVSCKASGYTFTSNWMHWVKQRPGQGLEWIGEIDPSDSYTNYNQKFKGKAKLTVDKSSSTAYMEVSDLTAEDSAVYYCARGSNPYYYAMDYWGQGTSVTVSS

SEQ ID NO: 17 - protein, 120 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 18 - protein, 120 aa, synthetic construct
QVQLEQPGAEVKKPGASVKVSCKASGYTFTSNWMHWVRQAPGKGLEWIGEIDPSDSYTNYNQKFDGKAKLTVDKSSSTAYMEVSDLTSEDSATYYCARGSNPYYYAMDYWGQGTSVTVSS

SEQ ID NO: 19 - protein, 120 aa, synthetic construct
QVQLEQPGAEVVKPGASVKVSCKTSGYTFTSNWMHWVKQRPGQGLEWIGEIDPSDSYTNYNQKFDGKAKLTVDKSSSTAYMEVSDLTSEDSATYYCARGSNPYYYAMDYWGQGTSVTVSS

SEQ ID NO: 20 - protein, 120 aa, synthetic construct
QVQLQQPGAEVKKPGASVRLSCETSGYTFTSNWMHWVKQRPDQGLEWIGEIDPSDSYTNYNQKFDGKAELTVDKSSSTAYMEVSDLTSEDSAVYYCARGSNPYYYAMDYWGQGTSVTVSS

SEQ ID NO: 21 - protein, 120 aa, synthetic construct
QVQLEQPGAEVKKPGASVKVSCKASGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 22 - protein, 29 aa, synthetic construct
QVQLXQPGAEVXKPGASVXXSCXXSGYTF

SEQ ID NO: 23 - protein, 14 aa, synthetic construct
WVXQXPXXGLEWIG

SEQ ID NO: 24 - protein, 38 aa, synthetic construct
YNQKFXGKAXLTVDKSSSTAYMEVSXLTXEDSAXYYCA

SEQ ID NO: 25 - protein, 104 aa, synthetic construct
QIVLTQSPAIMSASPGEKVTMTCSASSGVNYMHWYQQKPGTSPKRWIYDTSKLASGVPARFSGSGSGTSYSLTISSMEAEDAATYYCHQRGSYTFGGGTKLEIK

SEQ ID NO: 26 - protein, 104 aa, synthetic construct
QIALTQEPAIMSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 27 - protein, 104 aa, synthetic construct
DIVLTQSPATLSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 28 - protein, 104 aa, synthetic construct
QIVLTQSPATLSASPGEKATMTCSASSGANYMHWYQQKPGTSPKRWIYDTSKLASGVPARFSGSGSGTSYSLTIESMEAEDAATYYCHQRGSYTFGGGTKLEIK

SEQ ID NO: 29 - protein, 104 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIK

SEQ ID NO: 30 - protein, 104 aa, synthetic construct
QIALTQEPAIMSASPGEKATMTCSASSGVNYMHWYQQKPGTSPKRWIYDTSKLDSGVPARFSGSGSGTSYSLTIESMEAEDAATYYCHQRGSYTFGGGTKLEIK

SEQ ID NO: 31 - protein, 104 aa, synthetic construct
QIVLTQSPATLSASPGEKATMTCSASSGANYMHWYQQKPGTSPKRWIYDTDKTASGVPARFSGSGSGTSYSLTIESMEAEDAATYYCHQRGSYTFGGGTKLEIK

SEQ ID NO: 32 - protein, 23 aa, synthetic construct
XIXLTQXPAXXSASPGEKXTMTC

SEQ ID NO: 33 - skipped

SEQ ID NO: 34 - protein, 120 aa, synthetic construct; identical to SEQ ID NO: 13

SEQ ID NO: 35 - protein, 104 aa, synthetic construct; identical to SEQ ID NO: 25

SEQ ID NO: 36 - protein, 9 aa, synthetic construct
VRLSCKTSG

SEQ ID NO: 37 - protein, 9 aa, synthetic construct
WVKQRPGQG

SEQ ID NO: 38 - protein, 9 aa, synthetic construct
YNQKFKGKA

SEQ ID NO: 39 - protein, 9 aa, synthetic construct
FKGKAKLTV

SEQ ID NO: 40 - protein, 9 aa, synthetic construct
YMEVSSLTS

SEQ ID NO: 41 - protein, 9 aa, synthetic construct
VYYCARGSN

SEQ ID NO: 42 - protein, 9 aa, synthetic construct
IVLTQSPAI

SEQ ID NO: 43 - protein, 9 aa, synthetic construct
VLTQSPAIM

SEQ ID NO: 44 - protein, 9 aa, synthetic construct
VTMTCSASS

SEQ ID NO: 45 - protein, 9 aa, synthetic construct
VNYMHWYQQ

SEQ ID NO: 46 - protein, 9 aa, synthetic construct
WIYDTSKLA

SEQ ID NO: 47 - protein, 9 aa, synthetic construct
IYDTSKLAS

SEQ ID NO: 48 - protein, 13 aa, synthetic construct
CGTAAGTGGATCC

SEQ ID NO: 49 - protein, 12 aa, synthetic construct
GCTAGCTCCAGC

SEQ ID NO: 50 - protein, 450 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 450 aa, synthetic construct; identical to SEQ ID NO: 50

SEQ ID NO: 52 - protein, 211 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 211 aa, synthetic construct; identical to SEQ ID NO: 52

SEQ ID NO: 54 - protein, 450 aa, synthetic construct; identical to SEQ ID NO: 50

SEQ ID NO: 55 - protein, 211 aa, synthetic construct; identical to SEQ ID NO: 52

(19) United States
(12) Patent Application Publication
(10) Pub. No.: US 2026/0250383 A1
(43) Pub. Date: Aug. 27, 2026

(54) METHODS OF TREATMENT OF ITP

(71) Applicant: Climb Bio Operating Inc., Wellesley, MA (US)

(72) Inventors: Nishi Rampal, Wellesley, MA (US); Jan Hillson, Wellesley, MA (US); Aoife Brennan, Wellesley, MA (US); William Bonificio, Wellessley, MA (US); Stephen Thomas, Wellesley, MA (US)

(21) Appl. No.: 19/355,252

(22) Filed: Oct. 10, 2025

Related U.S. Application Data
(60) Provisional application No. 63/706,845, filed on Oct. 14, 2024.
(60) Provisional application No. 63/778,599, filed on Mar. 27, 2025.

(51) Int. Cl. C07K 16/28; A61K 39/00; A61P 7/04

(52) U.S. Cl. CPC C07K 16/2803; A61P 7/04; A61K 2039/505

(57) ABSTRACT

The invention provides novel methods for treatment of patients with immune thrombocytopenia (ITP) by administration of compositions comprising a therapeutic agent for the treatment of ITP. According to the invention, the therapeutic agent for the treatment of ITP may be an anti-CD19 antibody. The invention further provides novel dosing regimen for the treatment of patients with ITP.

### I. FIELD OF THE INVENTION

The field of the invention is directed to a method of treatment of immune thrombocytopenia (ITP) by administration of a composition comprising a therapeutic target.

### II. SEQUENCE LISTING

The present application is being filed along with a Sequence Listing XML in electronic format. The Sequence Listing XML is provided as a file named CLIMB-006-02US-Seq-Listing.xml, created on Oct. 9, 2025, which is 55,918 bytes in size. The Sequence Listing XML file and the information in the electronic format of the Sequence Listing XML file is incorporated herein by reference in its entirety.

### III. BACKGROUND

Immune thrombocytopenia (ITP) is an autoimmune disease. ITP is an illness that can lead to bruising and bleeding. In particular, antiplatelet autoantibodies lead to accelerated removal of platelets by macrophages with bone marrow compensation.

Low levels of the cells that help blood clot, also known as platelets, most often cause the bleeding. Once known as idiopathic thrombocytopenia purpura, ITP can cause purple bruises. It also can cause tiny reddish-purple dots on the skin that look like a rash. Children can get ITP after a virus. They most often get better without treatment. In adults, the illness often lasts months or years. People with ITP who aren't bleeding and whose platelet count isn't too low might not need treatment. For worse symptoms, treatment might include medicines to raise platelet count or surgery to remove the spleen.

### IV. SUMMARY OF THE INVENTION

The invention provides novel methods for treatment of immune thrombocytopenia (ITP). In certain aspects, the invention provides treating a subject having immune thrombocytopenia (ITP) comprising administering to the subject a composition comprising a therapeutically effective amount an anti-CD19 antibody. The anti-CD19 antibody may be a humanized anti-CD19 antibody. In certain embodiments, the anti-CD19 antibody comprises: a heavy chain variable region at least 95% identical to SEQ ID NO: 17 and/or a light chain variable region at least 95% identical to SEQ ID NO: 29. The invention beneficially provides that the anti-CD19 antibodies provide a good therapeutic response for patients with ITP. In particular, there are no previous therapies for ITP patients with compositions comprising anti-CD19 antibodies. The invention beneficially recognizes that the methods provided herein may provide a good response as compared to the previously known therapies because it targets CD-19+ plasma cells.

The methods of the invention may be for patients who have had a previous therapy for treatment and/or management of ITP. In certain embodiments, the patient may have not benefited from the previous treatment regimen. Alternatively, the patient may have relapsed after the previous treatment regimen.

In certain embodiments, the anti-CD19 antibody is budoprutug. In certain embodiments, the composition comprising the anti-CD19 antibody is administered parenterally. In certain embodiments, the composition is administered via an intravenous (IV) infusion.

In certain embodiments, the composition comprising the anti-CD19 antibody is administered in a dosing cycle. In certain embodiments, the dose cycle comprises administration of a single dose. In certain embodiments, the dose cycle comprises administration of more than one dose. In certain embodiments, the dose cycle comprises administration of two doses. In certain embodiments, the composition is administered at days 1 and 15 at the start of the treatment, wherein day 1 is the start of the treatment.

In certain embodiments, the composition is further administered at days 169 and 183 after the start of the treatment. In certain embodiments, the composition is administered in treatment cycles, each treatment cycle comprising administration of the composition at days 1 and 15 at start of each treatment cycle, wherein there is a 12 to 36-week interval between start of each said treatment cycle. In certain embodiments, interval between each treatment cycle is 12 weeks. In certain embodiments, said interval between each treatment cycle is 24 weeks. In certain embodiments, said interval between each treatment cycle is 36 weeks.

In certain embodiments, the composition comprises about 100 mg to about 500 mg of the anti-CD19 antibody. In certain embodiments, the composition comprises about 100 mg to about 400 mg of the anti-CD19 antibody. In certain embodiments, the composition comprises about 100 mg to about 300 mg of the anti-CD19 antibody. In certain embodiments, the composition comprises about 100 mg of the anti-CD19 antibody. In certain embodiments, the composition comprises about 200 mg of the anti-CD19 antibody. In certain preferred embodiments, the anti-CD19 antibody is budoprutug.

In certain embodiments, the composition comprises about 100 mg to about 1200 mg of the anti-CD19 antibody. In certain embodiments, the composition comprises about 100 mg to about 1000 mg of the anti-CD19 antibody. In certain embodiments, the composition comprises about 200 mg to about 100 mg of the anti-CD19 antibody. In certain embodiments, the composition comprises about 600 mg to about 100 mg of the anti-CD19 antibody. In certain embodiments, the composition comprises about 100 mg to about 500 mg of the anti-CD19 antibody. In certain embodiments, the composition comprises about 100 mg to about 300 mg of the anti-CD19 antibody.

In certain embodiments, the composition comprises about 100 mg of the anti-CD19 antibody. In certain embodiments, the composition comprises about 200 mg of the anti-CD19 antibody. In certain embodiments, the composition comprises about 250 mg of the anti-CD19 antibody. In certain embodiments, the composition comprises about 500 mg of the anti-CD19 antibody. In certain embodiments, the composition comprises about 600 mg of the anti-CD19 antibody. In certain embodiments, the composition comprises about 1000 mg of the anti-CD19 antibody. In certain preferred embodiments, the anti-CD19 antibody is budoprutug.

In certain embodiments, the composition comprises 100 mg of the anti-CD19 antibody. In certain embodiments, the composition comprises 200 mg of the anti-CD19 antibody. In certain embodiments, the composition comprises 250 mg of the anti-CD19 antibody. In certain embodiments, the composition comprises 500 mg of the anti-CD19 antibody. In certain embodiments, the composition comprises 600 mg of the anti-CD19 antibody. In certain embodiments, the composition comprises 1000 mg of the anti-CD19 antibody. In certain preferred embodiments, the anti-CD19 antibody is budoprutug.

In certain aspects, the method of the invention provides a novel “treat to target” approach for the treatment of ITP. This approach beneficially provides the measurement of the biological parameters associated with ITP after administration of the composition. The method of the invention provides the flexibility to alter the dosing regimen to provide additional doses of the anti-CD19 antibody after the initial dose to get optimal response for the treatment of ITP patients.

In certain embodiments, the methods of the invention further comprise measuring concentration for markers or biological parameters of ITP progression in the subject after the administration of the initial dose. The method further provides that an additional dosing cycle of the anti-CD19 antibody may be administered. In certain embodiments, the additional dosing cycle is administered if there is below expected response after the administration of the initial dosing cycle of the anti-CD19 antibody.

The markers or biological parameters for measurement of ITP progression could be measuring concentration of any marker or parameter associated to monitor the progression of ITP or symptoms thereof. The markers or parameters could be any parameters used to measure the efficacy of certain treatments. In certain embodiments, the methods of the invention provide that the markers are selected from the group consisting of platelet count and B cells. The concentration of B-cells measured could be circulating B cells.

In certain embodiments, the additional dosing cycle is administered if the markers of ITP progression demonstrate insufficient response after administration of initial dose of the composition. The timeline for measurement of markers or biological parameters could be determined based on the expected timeline for seeing a response after the administration of the initial dose of the composition comprising anti-CD19 antibody.

In certain embodiments, the measuring step is conducted about 8 to about 16 weeks after administration of initial dose of the composition comprising anti-CD19 antibody. In certain embodiments, the measuring step is conducted about at least 12 weeks after administration of initial dose of the composition comprising anti-CD19 antibody. In certain embodiments, the measuring step is conducted about at least 12 weeks after administration of initial dose of the composition comprising anti-CD19 antibody.

The invention recognizes that an additional dose of the treatment with anti-CD19 antibodies may be administered if an inadequate response is observed after the initial dose. The parameters or markers may be measured at the discretion of the physician treating the condition in the patient. The timeline for measurement of the markers of biological parameters may account for each patient's physiological condition. For example, the parameters monitored are the platelet count and/or circulating B-cells in the patient. In certain embodiments, the additional dose is administered if the platelet count is less than 30,000/μL at least 12 weeks after first dosing cycle. In certain embodiments, the additional dosing cycle is administered if the B-cell count is more than 40/μL at least 12 weeks after first dosing cycle.

In certain embodiments, the additional dosing cycle comprises administration of two doses of the composition comprising anti-CD19 antibody. In certain embodiments, the additional dosing cycle comprises administration of a single dose of the composition comprising anti-CD19 antibody.

In certain embodiments, the additional dosing cycle comprises administration of a single dose comprising about 100 mg to about 1000 mg of the anti-CD19 antibody. In certain embodiments, the additional dosing cycle comprises administration of a single dose comprising about 50 mg to about 500 mg of the anti-CD19 antibody. In certain embodiments, the additional dosing cycle comprises administration of a single dose comprising about 100 mg to about 400 mg of the anti-CD19 antibody. In certain embodiments, the additional dosing cycle comprises administration of a single dose comprising about 100 mg to about 300 mg of the anti-CD19 antibody. In certain embodiments, the additional dosing cycle comprises administration of a single dose comprising about 100 mg to about 200 mg of the anti-CD19 antibody. In certain embodiments, the additional dosing cycle comprises administration of a single dose comprising about 100 mg of the anti-CD19 antibody. In certain embodiments, the additional dosing cycle comprises administration of a single dose comprising about 200 mg of the anti-CD19 antibody. In certain embodiments, the additional dosing cycle comprises administration of a single dose comprising about 600 mg of the anti-CD19 antibody. In certain embodiments, the additional dosing cycle comprises administration of a single dose comprising about 1000 mg of the anti-CD19 antibody.

In certain embodiments, the additional dosing cycle comprises administration of two doses, wherein at least one two doses comprises from about 100 mg to about 1000 mg of the anti-CD19 antibody. In certain embodiments, the additional dosing cycle comprises administration of two doses, wherein at least one two doses comprises from about 50 mg to about 500 mg of the anti-CD19 antibody. In certain embodiments, the additional dosing cycle comprises administration of two doses, wherein at least one two doses comprises from about 100 mg to about 400 mg of the anti-CD19 antibody. In certain embodiments, the additional dosing cycle comprises administration of two doses, wherein at least one two doses comprises from about 100 mg to about 300 mg of the anti-CD19 antibody. In certain embodiments, the additional dosing cycle comprises administration of two doses, wherein at least one two doses comprises from about 100 mg to about 200 mg of the anti-CD19 antibody. In certain embodiments, the additional dosing cycle comprises administration of two doses, wherein at least one two doses comprises from about 100 mg of the anti-CD19 antibody. In certain embodiments, the additional dosing cycle comprises administration of two doses, wherein at least one two doses comprises about 200 mg of the anti-CD19 antibody. In certain preferred embodiments, the anti-CD19 antibody is budoprutug. In certain embodiments, the additional dosing cycle comprises administration of two doses, wherein at least one two doses comprises about 600 mg of the anti-CD19 antibody. In certain preferred embodiments, the anti-CD19 antibody is budoprutug. In certain embodiments, the additional dosing cycle comprises administration of two doses, wherein at least one two doses comprises about 1000 mg of the anti-CD19 antibody. In certain preferred embodiments, the anti-CD19 antibody is budoprutug.

In certain embodiments, the additional dosing cycle comprises administration of two doses, wherein at least one two doses comprises about 100 mg to about 1200 mg of the anti-CD19 antibody. In certain embodiments, the additional dosing cycle comprises administration of two doses, wherein at least one two doses comprises about 100 mg to about 1000 mg of the anti-CD19 antibody. In certain embodiments, the additional dosing cycle comprises administration of two doses, wherein at least one two doses comprises about 200 mg to about 1000 mg of the anti-CD19 antibody. In certain embodiments, the additional dosing cycle comprises administration of two doses, wherein at least one two doses comprises about 200 mg to about 300 mg of the anti-CD19 antibody. In certain embodiments, the additional dosing cycle comprises administration of two doses, wherein at least one two doses comprises about 250 mg of the anti-CD19 antibody. In certain embodiments, the additional dosing cycle comprises administration of two doses, wherein at least one two doses comprises about 500 mg of the anti-CD19 antibody. In certain embodiments, the additional dosing cycle comprises administration of two doses, wherein at least one two doses comprises about 600 mg of the anti-CD19 antibody. In certain embodiments, the additional dosing cycle comprises administration of two doses, wherein at least one two doses comprises about 1000 mg of the anti-CD19 antibody.

In certain embodiments, the invention provides pre-medication prior to each administration of the anti-CD19 antibody. In certain embodiments, the invention provides pre-medication prior to the infusion of budoprutug to reduce the frequency and severity of infusion-related reaction (IRRs). The premedication could be with a medical auxiliary product. In certain embodiments, the premedication is with a corticosteroid, antihistamine, or an antipyretic. In certain embodiments, the corticosteroid is administered intravenously. In certain embodiments, the antihistamine is administered orally. In certain embodiments, the antipyretic is administered orally.

In certain preferred embodiments, the corticosteroid may be methylprednisone or an equivalent thereof. In certain preferred embodiments, the antihistamine may be diphenhydramine or an equivalent thereof. In certain preferred embodiments, the antipyretic may be acetaminophen/paracetamol or an equivalent thereof.

In certain aspects, the invention provides a method of treating a subject having immune thrombocytopenia (ITP), the method comprising:
administering a first cycle of a pharmaceutical composition comprising a therapeutically effective amount of a therapeutic agent;
monitoring markers associated with ITP progression in the subject; and
providing a second cycle of compositions comprising therapeutically effective amount of said anti-CD19 antibody if the biological parameters demonstrate an inadequate physiological response after the first cycle.

In certain embodiments, the therapeutic agent is an anti-CD19 antibody. In certain embodiments, the second cycle is different from the first cycle. In certain embodiments, the first cycle comprises administration at days 1 and 15 at the start of the treatment. In certain embodiments, the markers are selected from the group consisting of platelet count and B cells. In certain embodiments, the monitoring step is conducted about 8 to about 16 weeks after start of the first cycle. In certain embodiments, the monitoring step is conducted about 12 weeks after start of the first cycle. In certain embodiments, the second cycle is administered if the platelet count is less than 30,000/μL at least 12 weeks after start of the first cycle. In certain embodiments, the second cycle is administered if the B-cell count is more than 40/μL at least 12 weeks after start of the first cycle. In certain embodiments, the subject has insufficient response to at least one prior therapy for treatment of ITP.

The invention beneficially recognizes that the methods provided herein demonstrate treatment of orphan conditions, such as ITP, in a personalized medicine approach. CD19 B cell depletion has the potential to reshape the treatment in immune mediated diseases, and the unique treat to target approach provided herein enables the treatment of patients throughout their journey and provide a tailored approach for each patient.

### V. BRIEF DESCRIPTION OF THE DRAWINGS

FIG. 1 provides an overview of the current methods of management and administered therapies to patients with ITP.

FIG. 2 provides the data pertaining to plasma cells in spleen after treatment with rituximab.

FIG. 3 provides a schematic of the mechanism for the B cell population growth after treatment with rituximab.

FIG. 4 and FIG. 5 provide overview of Phase 2 trial design for treatment of ITP.

### VI. DETAILED DESCRIPTION

### Anti-CD19 Antibodies of the Invention:

In certain aspects, the invention provides anti-CD19 antibodies which feature an amino acid sequence defining a modified immunoglobulin heavy chain framework region comprising amino acid residues 1-30 of SEQ ID NO:22, wherein one or more of the amino acid residues at positions X5, X12, X19, X20, X23, and X24 are as follows: X5 is Q or E, X12 is V or K, X19 is R or K, X20 is L or V, X23 is K, E or D, or X24 is T or A. In certain embodiments, at least one of the amino acid residues at positions X5, X12, X19, X20, X23, or X24 is not the same amino acid residue as the amino acid at the corresponding position in the unmodified immunoglobulin heavy chain framework region as set forth in amino acid residues 1-30 of SEQ ID NO: 13. In one embodiment, X23 is E or D.

In certain embodiments, the anti-CD19 antibodies feature an amino acid sequence defining a modified immunoglobulin heavy chain framework region comprising amino acid residues 1-14 of SEQ ID NO:23, wherein one or more of the amino acid residues at positions X3, X5, X7, and X8, are as follows: X3 is K or R, X5 is R, T, or A, X7 is G, D, or E, or X8 is Q or K. According to this aspect of the invention, at least one of the amino acid residues at positions X3, X5, X7, or X8 is not the same as the amino acid at the corresponding position in the unmodified immunoglobulin heavy chain framework region as set forth in amino acid residues 36-49 of SEQ ID NO: 13. In one embodiment, X7 is E or D.

In certain embodiments, the anti-CD19 antibodies feature an amino acid sequence defining a modified immunoglobulin heavy chain framework region comprising amino acid residues 1-39 of SEQ ID NO:24, wherein one or more of the amino acid residues at positions X6, X10, X26, X29, and X34 are as follows: X6 is K, D, or E, X10 is K, E, or D, X26 is S, D, or E, X29 is S or A, or X34 is V or T. According to this aspect of the invention, at least one of the amino acid residues at positions X6, X10, X26, X29, or X34 is not the same as the amino acid at the corresponding position in the unmodified immunoglobulin heavy chain framework region as set forth in amino acid residues 60-98 of SEQ ID NO:13. In one embodiment, X10 is E or D.

In certain embodiments, the anti-CD19 antibodies feature an amino acid sequence defining a modified immunoglobulin light chain framework region comprising amino acid residues 1-23 of SEQ ID NO:32, wherein one or more of the amino acid residues at positions X1, X3, X7, X10, X11, and X19 are as follows: X1 is Q or D, X3 is V or A, X7 is S or E, X10 is I or T, X11 is M or L, or X19 is V or A. According to this aspect of the invention, at least one of the amino acid residues at positions X1, X3, X7, X10, X11, or X19 is not the same as the amino acid at the corresponding position in the unmodified immunoglobulin light chain framework region as set forth in amino acid residues 1-23 of SEQ ID NO:25. In one embodiment, X3 is A and X7 is E. In another embodiment, X1 is D, X10 is I, and X11 is L.

In certain embodiments, the invention features an amino acid sequence defining a modified immunoglobulin light chain complementarity determining region comprising amino acid residues 24-33 of SEQ ID NO:28.

In certain embodiments, the invention features an amino acid sequence defining a modified immunoglobulin light chain framework region comprising amino acid residues 56-87 of SEQ ID NO:28.

According to another aspect, the invention features an antibody variable region comprising an amino acid sequence selected from the group consisting of SEQ ID NO: 14, SEQ ID NO: 15, SEQ ID NO: 16, SEQ ID NO: 17, SEQ ID NO: 20, SEQ ID NO: 21, SEQ ID NO: 26, SEQ ID NO: 27, SEQ ID NO: 28, SEQ ID NO: 29, SEQ ID NO: 30, or SEQ ID NO: 31, wherein the antibody variable region specifically binds to CD19.

In certain embodiments, the invention features a polypeptide at least 90% or at least 95% identical to a B4 antibody heavy chain variable region, the polypeptide comprising an amino acid substitution at one or more residues corresponding to Val12, Leu20, Lys23, Thr24, Lys38, Gly42, Gln43, Lys65, Lys69, Ser85, Ser88, or Val93. In one embodiment, the polypeptide comprises one or more of substitutions Gln5Glu, Val12Lys, Arg19Lys, Leu20Val, Lys23Glu, Lys23 Asp, Thr24Ala, Lys38Arg, Arg40Thr, Gly42Asp, Gly42Glu, Gln43Lys, Lys65Asp, Lys65Glu, Lys69Glu, Lys69Asp, Ser85Asp, Ser85Glu, Ser88Ala, or Val93Thr.

According to another aspect, the invention features a polypeptide at least 90% or at least 95% identical to a B4 antibody light chain variable region, the polypeptide comprising an amino acid substitution at one or more residues corresponding to Val3, Ser7, Ile10, Met11, Val19, Val29, Ser51, Leu53, Ala54, or Ser75. In one embodiment, the polypeptide comprises one or more of substitutions Gln1Asp, Val3 Ala, Ser7Glu, Ile10Thr, Met11Leu, Val19Ala, Val29Ala, Ser51Asp, Leu53Thr, Ala54Asp, or Ser75Glu.

In certain aspects, the invention provides a process for preparation of anti-CD19 antibody, wherein the anti-CD19 antibody comprises variable domain comprising a light chain variable region that is at least 90% identical to SEQ ID NO: 25 and has an amino acid substitution at one or more residues corresponding to Ile10, Met11, Val19, Ser51, and Leu53; and the process comprises production of the anti-CD19 antibody in Chinese Hamster Ovary (CHO) cells.

In certain aspects, the invention provides a process for preparation of an anti-CD19 antibody, wherein: the anti-CD19 antibody comprises SEQ ID NO: 13 with an amino acid substitution at one or more residues corresponding to Gln5, Arg19, Leu20, Arg40, Gln43, Lys65, Ser85, Ser88, and Val93, and SEQ ID NO: 25 with an amino acid substitution at one or more residues corresponding to Ile10, Met11, Val19, Ser51, and Leu53; and the process comprises production of the anti-CD19 antibody in Chinese Hamster Ovary (CHO) cells.

In certain embodiments, the anti-CD19 antibody variable domain comprises a heavy chain variable region of SEQ ID NO: 17 and a light chain variable region of SEQ ID NO: 29. In certain embodiments, the anti-CD19 antibody is provided in U.S. Pat. No. 8,691,952, which is incorporated by reference in its entirety. In certain embodiments, the anti-CD19 antibody variable domain comprises a heavy chain variable region of SEQ ID NO: 17 and a light chain variable region of SEQ ID NO: 29 are provided in U.S. Pat. No. 8,691,952.

In certain embodiments, the heavy chain variable region of the anti-CD19 antibody has, compared to SEQ ID NO:13, one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, the heavy chain variable region of the anti-CD19 antibody is SEQ ID NO: 13 comprising an amino acid substitution at one or more residues corresponding to Gln5, Arg19, Leu20, Arg40, Gln43, Lys65, Ser85, Ser88, and Val93.

In certain embodiments, the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93 Thr.

In certain embodiments, wherein the heavy chain variable region of the anti-CD19 antibody is the amino acid sequence of SEQ ID NO: 17.

In certain embodiments, the anti-CD19 antibody variable domain of the anti-CD19 antibody further comprises a light chain variable region that is at least 90% identical to SEQ ID NO: 25 and has an amino acid substitution at one or more residues corresponding to Ile10, Met11, Val19, Ser51, and Leu53.

In certain embodiments, wherein the light chain variable region the anti-CD19 antibody has one or more amino acid substitutions selected from the group consisting of Ile10Thr, Met11Leu, Val19Ala, Ser51Asp, and Leu53Thr.

In certain embodiments, the light chain variable region is at least 95% identical to SEQ ID NO: 25.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is SEQ ID NO: 25 comprising an amino acid substitution at one or more residues corresponding to Ile10, Met11, Val19, Ser51, and Leu53.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is the amino acid sequence of SEQ ID NO: 29.

In certain embodiments, the heavy chain variable region comprises one or more of substitutions Gln5Glu, Arg19Lys, Leu20Val, Arg40Thr, Gln43Lys, Lys65Asp, Ser85Asp, Ser88Ala, and Val93Thr.

In certain embodiments, wherein the light chain variable region comprises one or more of substitutions Ile10Thr, Met11Leu, Val19Ala, Ser51Asp, and Leu53Thr.

In certain embodiments, the anti-CD19 antibody is VB119. In certain embodiments, VB119 is an anti-CD19, IgG1 monoclonal antibody. VB119 demonstrates antibody-dependent cell mediated toxicity (ADCC) with minimal to no complement dependent cytotoxicity (CDC).

In certain embodiments, the anti-CD19 antibody comprises one or more of the below listed subunits in the anti-CD19 antibody:

Subunit 1 (SEQ ID NO: 50):
QVQLEQPGAEVVKPGASVKVSCKTSGYTFTSNWMHWVKQTPGKGLEWIGEIDP
SDSYTNYNQKFDGKAKLTVDKSSSTAYMEVSDLTAEDSATYYCARGSNPYYYAMDYW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Subunit 2 (SEQ ID NO: 51):
QVQLEQPGAEVVKPGASVKVSCKTSGYTFTSNWMHWVKQTPGKGLEWIGEIDP
SDSYTNYNQKFDGKAKLTVDKSSSTAYMEVSDLTAEDSATYYCARGSNPYYYAMDYW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Subunit 3 (SEQ ID NO: 52):
QIVLTQSPATLSASPGEKATMTCSASSGVNYMHWYQQKPGTSPKRWIYDTDKTAS
GVPARFSGSGSGTSYSLTISSMEAEDAATYYCHQRGSYTFGGGTKLEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
Subunit 4 (SEQ ID NO: 53):
QIVLTQSPATLSASPGEKATMTCSASSGVNYMHWYQQKPGTSPKRWIYDTDKTAS
GVPARFSGSGSGTSYSLTISSMEAEDAATYYCHQRGSYTFGGGTKLEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC

In certain embodiments, the heavy chain sequence of the anti-CD19 antibody is provided below (SEQ ID NO: 54):

QVQLEQPGAEVVKPGASVKVSCKTSGYTFTSNWMHWVKQTPGKGLEWIGEIDP
SDSYTNYNQKFDGKAKLTVDKSSSTAYMEVSDLTAEDSATYYCARGSNPYYYAMDYW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK

In certain embodiments, the light chain sequence of the anti-CD19 antibody is provided below (SEQ ID NO: 55):

QIVLTQSPATLSASPGEKATMTCSASSGVNYMHWYQQKPGTSPKRWIYDTDKTAS
GVPARFSGSGSGTSYSLTISSMEAEDAATYYCHQRGSYTFGGGTKLEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC

In certain embodiments, the anti-CD19 antibody is budoprutug. In certain embodiments, budoprutug comprises a heavy chain and a light chain. In certain embodiments, budoprutug comprises heavy chain and light chain provided in SEQ ID NO: 54 and SEQ ID NO: 55 respectively.

In certain embodiments, the heavy chain of anti-CD19 antibody is at least 90% identical to SEQ ID NO: 54. In certain embodiments, the heavy chain of anti-CD19 antibody is at least 95% identical to SEQ ID NO: 54. In certain embodiments, the heavy chain of anti-CD19 antibody is at least 98% identical to SEQ ID NO: 54. In certain embodiments, the heavy chain of anti-CD19 antibody is identical to SEQ ID NO: 54.

In certain embodiments, the light chain of anti-CD19 antibody is at least 90% identical to SEQ ID NO: 55. In certain embodiments, the light chain of anti-CD19 antibody is at least 95% identical to SEQ ID NO: 55. In certain embodiments, the light chain of anti-CD19 antibody is at least 98% identical to SEQ ID NO: 55. In certain embodiments, the light chain of anti-CD19 antibody is identical to SEQ ID NO: 55.

### Fc Portion

In certain embodiments, the antibody variable domains of the antibodies prepared using the methods of the invention are optionally fused to an Fc portion. As used herein, the Fc portion encompasses domains derived from the heavy chain constant region of an immunoglobulin, preferably a human immunoglobulin, including a fragment, analog, variant, mutant or derivative of the constant region. The constant region of an immunoglobulin heavy chain is defined as a naturally-occurring or synthetically produced polypeptide homologous to at least a portion of the C-terminal region of the heavy chain, including the CH1, hinge, CH2, CH3, and, for some heavy chain classes, CH4 domains. The “hinge” region joins the CH1 domain to the CH2-CH3 region of an Fc portion. The constant region of the heavy chains of all mammalian immunoglobulins exhibit extensive amino acid sequence similarity.

In the present invention, the Fc portion typically includes at least a CH2 domain. For example, the Fc portion can include the entire immunoglobulin heavy chain constant region (CH1-hinge-CH2-CH3). Alternatively, the Fc portion can include all or a portion of the hinge region, the CH2 domain and the CH3 domain.

The constant region of an immunoglobulin is responsible for many important antibody effector functions, including those mediated by Fc receptor (FcR) binding and by complement binding. There are five major classes of the heavy chain constant region, classified as IgA, IgG, IgD, IgE, and IgM, each with characteristic effector functions designated by isotype.

IgG, for example, is separated into four γ isotypes: γ1, γ2, γ3, and γ4, also known as IgG1, IgG2, IgG3, and IgG4, respectively. IgG molecules can interact with multiple classes of cellular receptors including three classes of Fcγ receptors (FcγR) specific for the IgG class of antibody, namely FcγRI, FcγRII, and FcγRIII. The sequences important for the binding of IgG to the FcγR receptors have been reported to be in the CH2 and CH3 domains.

It is also often useful to alter the serum half-life of the antibody. The serum half-life of an antibody, as of an immunoglobulin fusion protein, is influenced by the ability of that antibody to bind to an Fc receptor (FcR) (Gillies et al., Cancer Research (1999) 59:2159-66). The CH2 and CH3 domains of IgG2 and IgG4 have undetectable or reduced binding affinity to Fc receptors compared to those of IgG1. Accordingly, the serum half-life of the featured antibody can be increased by using the CH2 and/or CH3 domain from IgG2 or IgG4 isotypes. Alternatively, the antibody can include a CH2 and/or CH3 domain from IgG1 or IgG3 with modification in one or more amino acids in these domains to reduce the binding affinity for Fc receptors (see, e.g., U.S. patent application Ser. No. 09/256,156, published as U.S. patent application publication 2003-0105294).

In certain embodiments, an Fc portion fused to an antibody variable region of the invention can contain CH2 and/or CH3 domains and a hinge region that are derived from different antibody isotypes. For example, the Fc portion can contain CH2 and/or CH3 domains of IgG2 or IgG4 and a hinge region of IgG1. Assembly of such hybrid Fc portions has been described in U.S. patent application publication 2003-0044423.

When fused to an antibody variable region of the invention, the Fc portion may contain one or more amino acid modifications that generally extend the serum half-life of an Fc fusion protein. Such amino acid modifications include mutations substantially decreasing or eliminating Fc receptor binding or complement fixing activity. For example, one type of such mutation removes the glycosylation site of the Fc portion of an immunoglobulin heavy chain. In IgG1, the glycosylation site is Asn297 (see, for example, U.S. patent application Ser. No. 10/310,719, published as U.S. patent application publication 2003-0166163).

### Immune Thrombocytopenia

Immune thrombocytopenia (ITP) is an autoimmune disease. ITP is an illness that can lead to bruising and bleeding. In particular, antiplatelet autoantibodies lead to accelerated removal of platelets by macrophages with bone marrow compensation. The symptoms of ITP include bruising, bleeding episodes, and fatigue. The diagnosis relies on low platelet count, and is further supported by additional blood tests, i.e., CBC and blood smear, and antiplatelet antibody test. The diagnosis may further rely on bone marrow aspiration, if needed.

ITP is a predominantly an IgG1 immune mediated disorder with antiplatelet antibodies. These autoantibodies attach themselves to the platelet surface marking the cells for the immune system, targeting both their production and circulation. This results in shorter platelet lifespan with splenic destruction as a hallmark feature. The bone marrow attempts to compensate by increasing cell production, but the cascading immune disorders leaves patients vulnerable to the effects of thrombocytopenia. For patients this low platelet count results in bruising or petechiae and purpura, hemorrhagic episodes or extensive bleeding, and chronic fatigue. Children fare better than adults with the natural history of the disease taking a more benign course. Conversely, adults rarely have spontaneous remission; they are reliant on a number of treatments which contribute to higher mortality than is observed in aged-matched controls. In the US alone over 80,000 adults live with chronic ITP and they face a complex treatment journey. In fact, the majority of these patients relapse after receiving first line therapy with subsequent lines of therapy only partially addressing patient needs.

In general, most of the children have spontaneous remission within a few weeks or months. While adults often stabilize after the first line therapy, the majority eventually relapse or become refractory, necessitating treatment with second and often third line therapies. In certain hard to treat situations, splenectomy is also considered as an option for treatment.

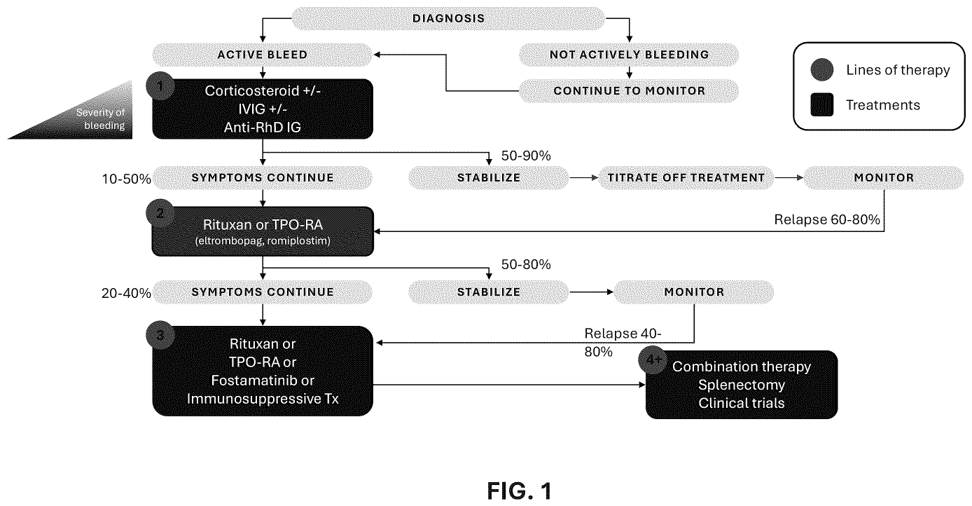

An overview of the current methods of management of ITP is provided in FIG. 1. For example, the current treatment includes treatment with corticosteroid, intravenous immunoglobulin (IVIG), and anti-RhD immunoglobulin. The International Working Group (IWG) on ITP and the American Society of Hematology (ASH) 2019 guidelines.

Based on the current treatment recommendations of the American Society of Hematology, it is clear that the there is no efficacious treatment for ITP patients. There is a clear significant unmet need for treatment of these patients. Even if a patient is fortunate enough to stabilize on first line therapy, upwards of 80% relapse and move to 2^{nd} line treatments, namely Rituximab or thrombopoietin receptor agonists. Many of those necessitate a 3^{rd} line treatment with additional doses of Rituximab and thrombopoietin receptor agonists, or a trial of Fostamatinib or other ISTs. If unsuccessful, combination therapy and even splenectomy are considered. A more detailed examination of the current treatments and their mechanism of action reveals modalities that attempt to intervene down stream in disease pathogenesis. Anti-RhD IG a first line therapy and Fostamatinib a third line therapy attempt to evade the already cascading immune mediated platelet destruction. Other treatments like TPO-RA try to boost bone marrow production of platelets. Few current treatments target upstream disease pathogenesis with Rituximab attempting to target pathological B-cells but falling short. Certainly, rituximab or CD20 B cell depleters are effective, but they can only address a limited B cell lineage. Most importantly, plasma cells, the autoantibody producing cells implicated in ITP, are not addressed by rituximab or CD 20 depleters, a key feature that Steven already highlighted.

This fundamental shortfall may be overcome by the methods of the current invention. The invention beneficially realizes that anti-CD19 antibodies may provide an impact by targeting plasma cells and plasmablasts in addition to other cells across the B cell lineage.

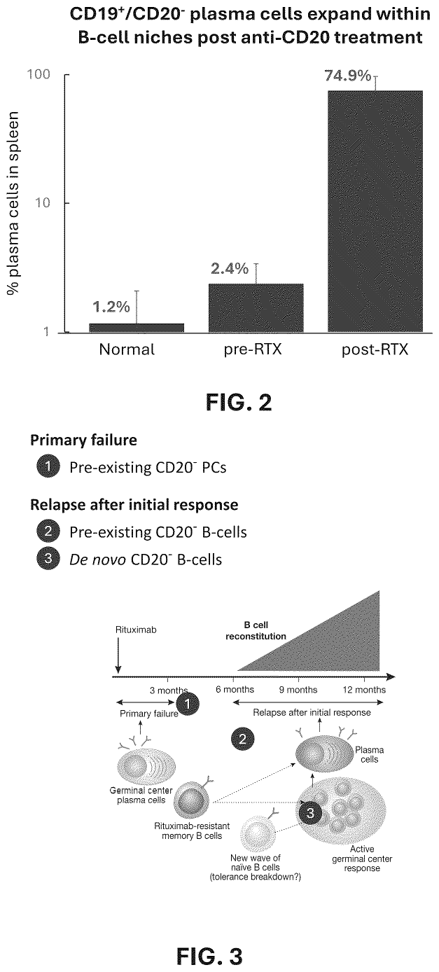

ITP patients are unlikely to respond to therapies with anti-CD20 agents, such as rituximab, because ITP patients have CD19+/CD20″ cells. The data provided in FIG. 2 demonstrates that CD19+/CD20-plasma cells expand within B-cell niches post anti-CD20 treatment. Moreover, FIG. 3 provides the mechanisms for primary failure of anti-CD19 antibodies. The primary failure could be due to pre-existing CD20″ plasma cells. In other mechanisms, there may also be a relapse after the initial response. This includes pre-existing CD20″ B-cells and de novo CD20-B-cells.

Rituximab depletes peripheral B cells but its limited effect leaves tissues vulnerable and the underlying disease only partially treated as evidenced by primary failures and more commonly high relapse rates. FIG. 2 and FIG. 3 demonstrate that CD20 negative B cells continue to drive disease through expansion and formation of germinal centers. The splenic plasma cells are also a big driver of the disease progression. In the pre Rituximab treated patient only 2.4% plasma cells are found in the spleen, which is in dramatic contrast to the post rituximab treated patient with nearly 75% plasma cells in the spleen. These pathogenic plasma cells, which are CD 20 negative and CD19 positive, continue to wreak havoc with persistent immune mediated destruction likely from tissue level expansion. Thus, the methods of the invention, which require administration of anti-CD19 antibody, target plasma cells as well as a number of cells across the B cell linage can potentially address this gap in the current treatment paradigm, not only serving as a more complete B cell depleter but addressing the rituximab failure population.

### Methods of the Invention

The invention provides novel methods for treatment of immune thrombocytopenia (ITP). In certain aspects, the invention provides treating a subject having immune thrombocytopenia (ITP) comprising administering to the subject a composition comprising a therapeutically effective amount an anti-CD19 antibody. The anti-CD19 antibody may be a humanized anti-CD19 antibody. In certain embodiments, the anti-CD19 antibody comprises: a heavy chain variable region at least 95% identical to SEQ ID NO: 17 and/or a light chain variable region at least 95% identical to SEQ ID NO: 29. The invention beneficially provides that the anti-CD19 antibodies provide a good therapeutic response for patients with ITP. In particular, there are no previous therapies for ITP patients with compositions comprising anti-CD19 antibodies. The invention beneficially recognizes that the methods provided herein may provide a good response as compared to the previously known therapies because it targets CD-19+ plasma cells.

The methods of the invention may be for patients who have had a previous therapy for treatment and/or management of ITP. In certain embodiments, the patient may have not benefited from the previous treatment regimen. Alternatively, the patient may have relapsed after the previous treatment regimen.

In certain embodiments, the anti-CD19 antibody is budoprutug. In certain embodiments, the composition comprising the anti-CD19 antibody is administered parenterally. In certain embodiments, the composition is administered via an intravenous (IV) infusion.

In certain embodiments, the composition comprising the anti-CD19 antibody is administered in a dosing cycle. In certain embodiments, the dose cycle comprises administration of a single dose. In certain embodiments, the dose cycle comprises administration of more than one dose. In certain embodiments, the dose cycle comprises administration of two doses. In certain embodiments, the composition is administered at days 1 and 15 at the start of the treatment, wherein day 1 is the start of the treatment.

In certain embodiments, the composition is further administered at days 169 and 183 after the start of the treatment. In certain embodiments, the composition is administered in treatment cycles, each treatment cycle comprising administration of the composition at days 1 and 15 at start of each treatment cycle, wherein there is a 12 to 36-week interval between start of each said treatment cycle. In certain embodiments, interval between each treatment cycle is 12 weeks. In certain embodiments, said interval between each treatment cycle is 24 weeks. In certain embodiments, said interval between each treatment cycle is 36 weeks.

In certain embodiments, the composition comprises about 100 mg to about 500 mg of the anti-CD19 antibody. In certain embodiments, the composition comprises about 100 mg to about 400 mg of the anti-CD19 antibody. In certain embodiments, the composition comprises about 100 mg to about 300 mg of the anti-CD19 antibody. In certain embodiments, the composition comprises about 100 mg of the anti-CD19 antibody. In certain embodiments, the composition comprises about 200 mg of the anti-CD19 antibody.

In certain embodiments, the composition comprises about 100 mg to about 500 mg of the anti-CD19 antibody. In certain embodiments, the composition comprises about 100 mg to about 400 mg of the anti-CD19 antibody. In certain embodiments, the composition comprises about 100 mg to about 300 mg of the anti-CD19 antibody. In certain embodiments, the composition comprises about 100 mg of the anti-CD19 antibody. In certain embodiments, the composition comprises about 200 mg of the anti-CD19 antibody. In certain preferred embodiments, the anti-CD19 antibody is budoprutug.

In certain embodiments, the composition comprises about 100 mg to about 1200 mg of the anti-CD19 antibody. In certain embodiments, the composition comprises about 100 mg to about 1000 mg of the anti-CD19 antibody. In certain embodiments, the composition comprises about 200 mg to about 100 mg of the anti-CD19 antibody. In certain embodiments, the composition comprises about 600 mg to about 100 mg of the anti-CD19 antibody. In certain embodiments, the composition comprises about 100 mg to about 500 mg of the anti-CD19 antibody. In certain embodiments, the composition comprises about 100 mg to about 300 mg of the anti-CD19 antibody.

In certain embodiments, the composition comprises about 100 mg of the anti-CD19 antibody. In certain embodiments, the composition comprises about 200 mg of the anti-CD19 antibody. In certain embodiments, the composition comprises about 250 mg of the anti-CD19 antibody. In certain embodiments, the composition comprises about 500 mg of the anti-CD19 antibody. In certain embodiments, the composition comprises about 600 mg of the anti-CD19 antibody. In certain embodiments, the composition comprises about 1000 mg of the anti-CD19 antibody. In certain preferred embodiments, the anti-CD19 antibody is budoprutug.

In certain aspects, the method of the invention provides a novel “treat to target” approach for the treatment of ITP. This approach beneficially provides the measurement of the biological parameters associated with ITP after administration of the composition. The method of the invention provides the flexibility to alter the dosing regimen to provide additional doses of the anti-CD19 antibody after the initial dose to get optimal response for the treatment of ITP patients.

In certain embodiments, the methods of the invention further comprise measuring concentration for markers or biological parameters of ITP progression in the subject after the administration of the initial dose. The method further provides that an additional dosing cycle of the anti-CD19 antibody may be administered. In certain embodiments, the additional dosing cycle is administered if there is below expected response after the administration of the initial dosing cycle of the anti-CD19 antibody.

The markers or biological parameters for measurement of ITP progression could be measuring concentration of any marker or parameter associated to monitor the progression of ITP or symptoms thereof. The markers or parameters could be any parameters used to measure the efficacy of certain treatments. In certain embodiments, the methods of the invention provide that the markers are selected from the group consisting of platelet count and B cells. The concentration of B-cells measured could be circulating B cells.

In certain embodiments, the additional dosing cycle is administered if the markers of ITP progression demonstrate insufficient response after administration of initial dose of the composition. The timeline for measurement of markers or biological parameters could be determined based on the expected timeline for seeing a response after the administration of the initial dose of the composition comprising anti-CD19 antibody.

In certain embodiments, the measuring step is conducted about 8 to about 16 weeks after administration of initial dose of the composition comprising anti-CD19 antibody. In certain embodiments, the measuring step is conducted about at least 12 weeks after administration of initial dose of the composition comprising anti-CD19 antibody. In certain embodiments, the measuring step is conducted about at least 12 weeks after administration of initial dose of the composition comprising anti-CD19 antibody.

The invention recognizes that an additional dose of the treatment with anti-CD19 antibodies may be administered if an inadequate response is observed after the initial dose. The parameters or markers may be measured at the discretion of the physician treating the condition in the patient. The timeline for measurement of the markers of biological parameters may account for each patient's physiological condition. For example, the parameters monitored are the platelet count and/or circulating B-cells in the patient. In certain embodiments, the additional dose is administered if the platelet count is less than 30,000/μL at least 12 weeks after first dosing cycle. In certain embodiments, the additional dosing cycle is administered if the B-cell count is more than 40/μL at least 12 weeks after first dosing cycle.

In certain embodiments, the additional dosing cycle comprises administration of two doses of the composition comprising anti-CD19 antibody. In certain embodiments, the additional dosing cycle comprises administration of a single dose of the composition comprising anti-CD19 antibody.

In certain embodiments, the additional dosing cycle comprises administration of a single dose comprising about 100 mg to about 1000 mg of the anti-CD19 antibody. In certain embodiments, the additional dosing cycle comprises administration of a single dose comprising about 50 mg to about 500 mg of the anti-CD19 antibody. In certain embodiments, the additional dosing cycle comprises administration of a single dose comprising about 100 mg to about 400 mg of the anti-CD19 antibody. In certain embodiments, the additional dosing cycle comprises administration of a single dose comprising about 100 mg to about 300 mg of the anti-CD19 antibody. In certain embodiments, the additional dosing cycle comprises administration of a single dose comprising about 100 mg to about 200 mg of the anti-CD19 antibody. In certain embodiments, the additional dosing cycle comprises administration of a single dose comprising about 100 mg of the anti-CD19 antibody. In certain embodiments, the additional dosing cycle comprises administration of a single dose comprising about 200 mg of the anti-CD19 antibody. In certain embodiments, the additional dosing cycle comprises administration of a single dose comprising about 600 mg of the anti-CD19 antibody. In certain embodiments, the additional dosing cycle comprises administration of a single dose comprising about 1000 mg of the anti-CD19 antibody.

In certain embodiments, the additional dosing cycle comprises administration of two doses, wherein at least one two doses comprises from about 100 mg to about 1000 mg of the anti-CD19 antibody. In certain embodiments, the additional dosing cycle comprises administration of two doses, wherein at least one two doses comprises from about 50 mg to about 500 mg of the anti-CD19 antibody. In certain embodiments, the additional dosing cycle comprises administration of two doses, wherein at least one two doses comprises from about 100 mg to about 400 mg of the anti-CD19 antibody. In certain embodiments, the additional dosing cycle comprises administration of two doses, wherein at least one two doses comprises from about 100 mg to about 300 mg of the anti-CD19 antibody. In certain embodiments, the additional dosing cycle comprises administration of two doses, wherein at least one two doses comprises from about 100 mg to about 200 mg of the anti-CD19 antibody. In certain embodiments, the additional dosing cycle comprises administration of two doses, wherein at least one two doses comprises from about 100 mg of the anti-CD19 antibody. In certain embodiments, the additional dosing cycle comprises administration of two doses, wherein at least one two doses comprises about 200 mg of the anti-CD19 antibody. In certain preferred embodiments, the anti-CD19 antibody is budoprutug. In certain embodiments, the additional dosing cycle comprises administration of two doses, wherein at least one two doses comprises about 600 mg of the anti-CD19 antibody. In certain preferred embodiments, the anti-CD19 antibody is budoprutug. In certain embodiments, the additional dosing cycle comprises administration of two doses, wherein at least one two doses comprises about 1000 mg of the anti-CD19 antibody. In certain preferred embodiments, the anti-CD19 antibody is budoprutug.

In certain embodiments, the additional dosing cycle comprises administration of two doses, wherein at least one two doses comprises about 100 mg to about 1200 mg of the anti-CD19 antibody. In certain embodiments, the additional dosing cycle comprises administration of two doses, wherein at least one two doses comprises about 100 mg to about 1000 mg of the anti-CD19 antibody. In certain embodiments, the additional dosing cycle comprises administration of two doses, wherein at least one two doses comprises about 200 mg to about 1000 mg of the anti-CD19 antibody. In certain embodiments, the additional dosing cycle comprises administration of two doses, wherein at least one two doses comprises about 200 mg to about 300 mg of the anti-CD19 antibody. In certain embodiments, the additional dosing cycle comprises administration of two doses, wherein at least one two doses comprises about 250 mg of the anti-CD19 antibody. In certain embodiments, the additional dosing cycle comprises administration of two doses, wherein at least one two doses comprises about 500 mg of the anti-CD19 antibody. In certain embodiments, the additional dosing cycle comprises administration of two doses, wherein at least one two doses comprises about 600 mg of the anti-CD19 antibody. In certain embodiments, the additional dosing cycle comprises administration of two doses, wherein at least one two doses comprises about 1000 mg of the anti-CD19 antibody.

In certain embodiments, the invention provides pre-medication prior to each administration of the anti-CD19 antibody. In certain embodiments, the invention provides pre-medication prior to the infusion of budoprutug to reduce the frequency and severity of infusion-related reaction (IRRs). The premedication could be with a medical auxiliary product. In certain embodiments, the premedication is with a corticosteroid, antihistamine, or a antipyretic. In certain embodiments, the corticosteroid is administered intravenously. In certain embodiments, the antihistamine is administered orally. In certain embodiments, the antipyretic is administered orally.

In certain preferred embodiments, the corticosteroid may be methylprednisone or an equivalent thereof. In certain preferred embodiments, the antihistamine may be diphenhydramine or an equivalent thereof. In certain preferred embodiments, the antipyretic may be acetaminophen/paracetamol or an equivalent thereof.

In certain aspects, the invention provides a method of treating a subject having immune thrombocytopenia (ITP), the method comprising:
administering a first cycle of a pharmaceutical composition comprising a therapeutically effective amount of a therapeutic agent;
monitoring markers associated with ITP progression in the subject; and
providing a second cycle of compositions comprising therapeutically effective amount of said anti-CD19 antibody if the biological parameters demonstrate an inadequate physiological response after the first cycle.

In certain embodiments, the therapeutic agent is an anti-CD19 antibody. In certain embodiments, the second cycle is different from the first cycle. In certain embodiments, the first cycle comprises administration at days 1 and 15 at the start of the treatment. In certain embodiments, the markers are selected from the group consisting of platelet count and B cells. In certain embodiments, the monitoring step is conducted about 8 to about 16 weeks after start of the first cycle. In certain embodiments, the monitoring step is conducted about 12 weeks after start of the first cycle. In certain embodiments, the second cycle is administered if the platelet count is less than 30,000/μL at least 12 weeks after start of the first cycle. In certain embodiments, the second cycle is administered if the B-cell count is more than 40/μL at least 12 weeks after start of the first cycle. In certain embodiments, the subject has insufficient response to at least one prior therapy for treatment of ITP.

The invention beneficially recognizes that the methods provided herein demonstrate treatment of orphan conditions, such as ITP, in a personalized medicine approach. CD19 B cell depletion has the potential to reshape the treatment in immune mediated diseases, and the unique treat to target approach provided herein enables the treatment of patients throughout their journey and provide a tailored approach for each patient.

### Phase 2 Trial ITP:

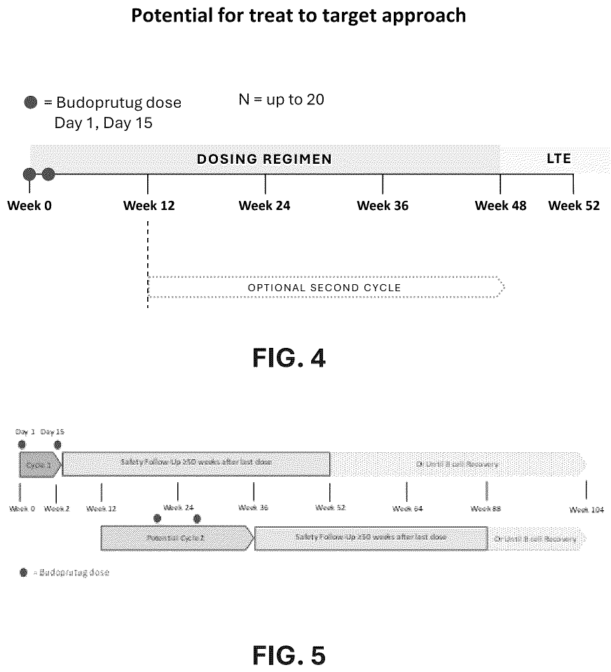

An overview of the ITP is provided in FIG. 4. The ITP phase 2 trial is a single arm open label study in adult patients with chronic disease evaluating the safety and efficacy of budoprutug.

It leverages learnings from prior ITP phase 2 trials where placebo rates have been historically very low. The primary endpoint will measure platelet improvement along with other disease parameters. However, truly unique to this study design is a ‘treat to target’ approach that allows redosing based on individual patient needs. Patients will receive a treatment cycle at day 1 and day 15 for those who meet eligibility criteria. Investigators can elect for a second cycle of treatment if improvement wanes, guided by individual patient laboratory findings and clinical needs. These parameters include low or falling platelet counts, that are deemed clinically significant, coupled with B cell counts. ITP offers an ideal serological marker in platelet count that reflects clinical picture. Additionally, since B cells return at a variable rate in each patient these 2 parameters taken together will allow providers to create the ‘just right’ treatment regimen to meet patients where they are. This personalized approach aims to optimize treatment efficacy. Individualized medicine for the individual living with a rare disease.

### EXAMPLES

Phase 1b/2a Open-Label Sequential-Cohort, Dose Escalation and Expansion Study to Evaluate Safety, Tolerability, Pharmacokinetics, Pharmacodynamics, and Preliminary Clinical Effectiveness of Budoprutug (TNT119) in Subjects with Immune Thrombocytopenia (ITP)

### Synopsis:

### Study Objectives:

### Primary Objective:

The primary objective of this study is:
To evaluate the safety and tolerability of ascending doses of budoprutug in subjects with ITP.

### Secondary Objectives:

The secondary objectives of this study include the following:
To investigate potential doses for subsequent dose-finding studies in subjects with ITP.
To characterize the pharmacokinetic (PK) profile of budoprutug in subjects with or ITP.
To evaluate the effects of budoprutug on B-cell depletion (pharmacodynamic [PD] response).

To evaluate the effects of budoprutug on platelet counts (ITP clinical response).

### Study Endpoints:

The primary safety endpoints include the following:
Incidence, relatedness, severity, and duration of treatment-emergent adverse events (TEAEs) and dose-limiting toxicities (DLTs).

The secondary endpoints include the following:
Budoprutug PK parameters (including area under the concentration-time curve, time to maximum observed concentration, terminal half-life, apparent clearance, and volume of distribution).
The change from baseline in absolute peripheral cluster of differentiation (CD) 20⁺ B-cell count.
The change in platelet count observed with budoprutug over time in subjects with ITP.
The percentage of subjects with ITP who achieve a stable, partial, and complete response by Week 12
The incidence of subjects who develop ADAs at any time after study drug administration.

The percentage of subjects on a steroid at baseline who are able to stop steroid treatment.

### Study Population:

This study will enroll adult male and female subjects who meet all of the inclusion criteria and none of the exclusion criteria. Subjects will be eligible for enrollment in this study regardless of race/ethnicity.

### Inclusion Criteria:

1. Willing and able to provide written informed consent.
2. Aged >18 years at the time of informed consent.
3. Diagnosed with primary ITP
4. Platelet count <30,000/μL despite an adequate trial of at least one prior therapeutic attempt. Platelet counts of <30,000/μL must be confirmed on 2 occasions at least 5 days apart, but no more than 14 days apart
5. Partial thromboplastin time <1.5× upper limit of normal (ULN), prothrombin time <1.5×ULN, total bilirubin <1.5×ULN unless due to Gilbert's syndrome, or an international normalized ratio <1.5 at screening. For subjects with Gilbert's syndrome, the limit for direct bilirubin is ≤ULN and for total bilirubin is ≤3×ULN.

### Exclusion Criteria:

1. CD19+ B-cell count <80 cells/μL at Screening or <40 cells/μL if B-cell depleting treatment was received from 24 weeks to 2 years prior to Screening, or if the subject has received or is receiving other immunosuppressive or immunomodulatory agent that may reduce circulating B cell levels.
2. Pregnant or lactating females.
3. Diagnosis of paroxysmal nocturnal hemoglobinuria, Evan's Syndrome, or any other bleeding disorder that could confound results and impact patient safety.
4. Prior treatment with rituximab or other B-cell depleting agents within 24 weeks prior to the first dose of study drug or plan to receive B-cell depleting agents during the study.
5. Current or planned treatment with any chronic anticoagulants or platelet aggregation-inhibiting drugs such as aspirin, nonsteroidal anti-inflammatory drugs, or thienopyridines within 14 days of planned dosing through the end of follow-up. Symptom-based intermittent dosing of nonsteroidal anti-inflammatory drugs is permitted.
6. Prior treatment with immunosuppressants (other than corticosteroids) within 30 days or 5 times the elimination half-life (whichever is longer) of the Screening Visit (e.g., calcineurin inhibitors, mycophenolate mofetil, azathioprine), or alkylating agents within 180 days of the Screening Visit.
7. Prior treatment with intravenous (IV) Ig within the last 90 days of the Screening Visit.
8. Active treatment for ITP other than steroids or TPO agonists within 30 days or 5 times the elimination half-life (whichever is longer) prior to the first dose of study drug without discussion with the Medical Monitor.
9. Treatment with any investigational drug within 30 days prior to the first dose of study drug, or at least 5 times the elimination half-life of the drug (whichever is longer), or plan to use an investigational device during the study.
10. History of malignancy within the past 5 years and likely to require chemotherapeutic or surgical treatment during the study, with the exception of non-melanoma skin cancer, cervical carcinoma in-situ, or ductal carcinoma-in-situ.
11. History of stem cell, bone marrow, or solid organ transplant.
12. Acute, chronic, or latent infection, including hepatitis B, hepatitis C or human immunodeficiency virus (HIV).
13. Evidence of active tuberculosis (TB) or being at high risk for TB based on:
a. History of active TB or untreated/incompletely treated latent TB. Subjects with a history of active or latent TB who have documentation of completion of treatment according to local guidelines and no evidence of ongoing TB may be enrolled.
b. History of recent (≤12 weeks of screening) close contact with someone with active TB (close contact is defined as ≥4 hours/week OR living in the same house.
14. Active or uncontrolled infection at the time of informed consent or study drug initiation.
15. Recent hospitalization for any reason within 14 days prior to Screening, unless approved by the Medical Monitor.
16. Receipt of a live vaccine within 28 days prior to the first dose of study drug or during the study. All other vaccines must be completed within 21 days prior to the first dose of study drug.

### Study Design and Duration:

This study is a Phase 1b/2a, open-label, sequential-cohort, dose escalation and expansion study to evaluate the safety, tolerability, PK, PD, and preliminary clinical effectiveness of budoprutug in subjects with ITP.

The study consists of the Dose Escalation phase 1b, and Dose Expansion phase 2a (Note: the dose expansion phase 2a will not be conducted in Georgia).

Subjects will be screened for study eligibility over 2 visits: the first visit on Days −21 to −14 (Screening) and the second visit on Days −13 to 0 (Qualifying Visit). The Qualifying Visit (Days −13 to 0) will occur at least 5 but no more than 14 days after the initial Screening Visit in order to confirm eligibility, particularly the platelet counts. After the Qualifying Visit, subjects who meet the eligibility criteria will be enrolled into the study to receive 2 doses of budoprutug on Day 1 and Day 15.

Each subject may receive additional budoprutug doses between Weeks 12 and 36 if certain safety and PD criteria are met.

All subjects will be followed for AEs and, if applicable, pregnancy, through 5 half-lives of budoprutug. In addition, subjects will be followed until there is evidence of recovery of circulating B cells. Specifically:
1. Subjects receiving a single cycle of budoprutug will undergo treatment and follow-up for a total of 52 weeks, including 2 weeks of treatment and 50 weeks of follow-up after the last budoprutug administration.
2. Subjects receiving a second cycle of budoprutug, initiated between Weeks 12 and 36, will undergo 50 weeks of follow-up after the last budoprutug administration. For example, subjects initiating the second treatment cycle at Week 12 (1 dose each at Weeks 12 and 14) will be followed until week 64, and subjects initiating the second cycle of budoprutug at Week 36 (2 doses at Weeks 36 and 38) will be followed until Week 88.
3. Subjects who have completed 50 weeks of follow-up after the last budoprutug administration but have not reached B cell recovery will continue to be followed for B cell recovery up to Week 104 of the study.

The schematic for study is provided in FIG. 5.
6 subjects enrolled sequentially in each cohort
Safety review committee review
Planned dose levels: 250, 500, 1000 mg biweekly (cumulative doses: 500, 1000, 2000 mg per dose cycle).

Phase 2a: up to 6 subjects enrolled at dose identified in Phase 1b.

### Dose Escalation Phase

During the Dose Escalation phase, subjects with ITP will be assigned to the currently open cohort in the study, per the table below. Each dosing cohort will be enrolled sequentially. Six subjects will be enrolled into each dosing cohort. The SRC will review all available data for the 6 subjects included in each cohort once the last dosed subject completes their Week 6 visit (4 weeks following the second dose of study drug in the initial dosing cycle). As the subject follow-up data are generated over time, platelet counts may also be factored into the evaluation. Dosing levels will continue sequentially to determine sufficient B-cell depletion and an acceptable safety and efficacy profile for subsequent trials.

### Doses of Budoprutug During the Dose Escalation Phase

| Dose | Dose | |
|---|---|---|
| Level | (mg) | Frequency |
| Level 1 | 250 | Every 14 days for 2 doses |
| (starting | | (500 mg cumulative dose) |
| dose) | | |
| Level 2 | 500 | Every 14 days for 2 doses |
| | | (1000 mg cumulative dose) |
| Level 3 | 1000 | Every 14 days for 2 doses |
| | | (2000 mg cumulative dose) |

In the Dose Escalation phase (Phase 1b), subjects will receive 2 bi-weekly doses of budoprutug at the specified dose level, administered via IV infusion. Dose escalation will be conducted using an adaptive design for each cohort, based on safety and B-cell depletion data (and any available platelet count clinical data). The first dose cohort will be 250 mg administered IV every 14 days for 2 doses (500 mg cumulative dose). The planned dose cohorts that will follow are anticipated to be 500 mg and 1000 mg administered IV every 14 days for 2 doses (1000 mg and 2000 mg cumulative dose, respectively). Each Dose Escalation cohort will be enrolled sequentially. The protocol will allow for intermediate doses or lower doses, based on the data generated in the study and recommendation of the SRC. Escalation doses between successive cohorts will not exceed more than double a previous cohort's cumulative dose. Following the first dosing cohort, the plan is flexible based on decisions of the SRC, focusing primarily on safety and PD effects of B-cell depletion. As the subject follow-up data are generated over time, platelet counts may also be factored into the evaluation.

For safety, if none of the initial 6 subjects experience a protocol-defined DLT during the initial treatment period, then enrollment into the next dose cohort can begin. If any of the subjects experience a DLT, action taken will vary depending on the event and the number of occurrences and will be guided by SRC recommendations. Please refer to the study stopping rules for details. For PD effects, if the data from 6 subjects do not demonstrate consistent effects, then up to 3 additional subjects can be enrolled at that dose level to better understand the PD effects prior to escalation to the next higher dose cohort.

### Dose Expansion Phase

The Dose Expansion phase (Phase 2a) will evaluate a cohort of subjects at the dose identified from Phase 1b. Up to 6 subjects in the expansion cohort will receive budoprutug at the dose identified for expansion and this cohort will also include subjects from the Dose Escalation phase treated with the expansion cohort dose.

Both the Dose Escalation and Dose Expansion phases will collect data on tolerability, PK, B-cell depletion, and complete blood counts (CBCs), including platelet counts.

### Additional Budoprutug Doses

Following the initial dosing cycle during the first 2 weeks of the study, the same subjects may receive additional budoprutug doses, i.e., 2 additional bi-weekly doses of budoprutug between Weeks 12 and 36. The additional dosing is intended for study subjects who have experienced some meaningful degree of clinical response but have not been able to maintain a complete remission and are therefore more likely to receive additional benefit. Subjects who achieve and maintain a complete remission and those who do not respond at all are unlikely to benefit from further dosing and therefore will not receive additional dosing. The additional dosing can be administered if criteria are met on the basis of immunologic or clinical response and the safety/tolerability profile as defined by the protocol. The additional budoprutug doses administered between Weeks 12 and 36 will match the dose level that is originally administered to each subject during the first cycle.

The additional dosing can be administered between 12 and 36 weeks if subjects satisfy the following criteria and who have not experienced any DLTs:
1. Demonstration of a clinical response to therapy at any time during the first 12 to 36 weeks of the study (defined as a platelet count >30,000/μL and a >25% platelet count increase from Baseline).
2. Circulating B-cells levels show evidence of recovery from the nadir achieved following the initial dose cycle.
3. Demonstrated loss of improvement between Weeks 12 to 36 on any of the following:
a. Platelet count has declined by 50% from the highest count following study drug administration;
b. Platelet count <30,000/μL;
c. Initiation or increase in corticosteroid rescue therapy to prevent further platelet decline and continuation of corticosteroids.

The additional dosing with budoprutug should also be reviewed and approved by the medical monitor prior to administration and all activities listed in the schedule of procedures should be completed.

The main goal of this study is to see how safe budoprutug is at different doses by monitoring the number and seriousness of side effects in participants. Other goals include understanding how the participant's body deals with budoprutug by checking the amount of budoprutug in the blood over time (this is called pharmacokinetics) and to find out the safe and effective dose of budoprutug. The study doctors will also see how effective budoprutug is in participants with ITP by measuring the change in platelet count and B-cells observed with budoprutug over time.

The participants are included in the stay if they:
are at least 18 years old;
have a specific platelet count;
have certain blood test results within specific limits, like white blood cell count, liver function tests, and kidney function tests; and/or
have taken corticosteroids or thrombopoietin (TPO) agonist, the dose must be stable for at least 14 days before starting budoprutug.

Participants will not be included in the study if they:
have B-cell count below certain levels;
have specific blood disorders like paroxysmal nocturnal hemoglobinuria, Evan's syndrome, or any other medical condition that may interfere with the study; and/or
have taken certain treatments like rituximab within the last 24 weeks or currently use, or recently used, certain medications that may interfere with the study or have been hospitalized for reason in the last 14 days.

The study has 2 parts. In Part 1, different sets of participants will receive increasing doses of budoprutug (dose escalation). Both the study doctor and participants will know the dose given. The first 6 participants will get the lowest dose. If no medical problems or side effects occur, the next 6 participants will receive the higher dose, and then again if the participants do not report any side effects, the next 6 participants will receive the highest dose. After finding a safe dose, this dose will be given to up to 6 more participants (dose expansion). Part 1 will be conducted in the following 3 periods over 23 clinic visits.

In Part 2, the long-term safety and B-cell recovery follow-up with budoprutug will be done for up to 52 weeks after Part 1 is completed. Participants will come for clinic visits every 4 weeks to check their B-cell levels and overall health. Follow-up will end once their B-cells return to normal, which may happen before the 52 weeks are over.

Overall, participants may be in the study for up to 2 years, including Part 1 and Part 2. They will answer some questions on how ITP affects their health and well-being. Blood and urine samples will be collected at most clinic visits. General health tests, physical exams, and electrocardiograms (tests that record the electrical activity of the heart) will be done. Certain substances called biomarkers in blood samples will be checked to see if budoprutug is effective in treating ITP. Participants will be asked to report any changes in their health to the study doctor or study staff.

Participants received budoprutug via an infusion into the vein of the arm.

Benefits/Risks of this Study:

Participants may or may not experience benefits from budoprutug. Like all medicines, budoprutug can cause unwanted effects called ‘side effects’. The potential safety risk of budoprutug to humans is not yet fully known. There is limited information about the possible side effects of budoprutug.

The most common side effects seen in 37 participants who took budoprutug in earlier studies are pain and redness at the injection site, feeling sick to the stomach, diarrhea, abdominal pain, vomiting, tiredness, chills, swelling in hands and feet, headache, dizziness, rash, cough, skin turns red, joint stiffness, decrease in certain type of white blood cells that increases the chances of infection in the participants, urinary tract and respiratory tract infection.

Drugs in the same category given in the vein are known to cause infusion-related reactions like delayed fever, rash, joint and muscle pain, blood or protein in urine, inflammation of body tissues, central nervous system complications, and a disorder in which red blood cells are destroyed faster than they can be made. Low oxygen levels may also occur. Other possible side effects are increased risk of serious infections, lower-than-normal levels of neutrophils (a type of white blood cell) in blood, and decrease in immunoglobulin (a protein in blood that help immune system fight off infections).

### Study Drug Dosing:

Participants will be enrolled in groups of approximately 6 participants each. The first group will receive the study drug at the initial (lower) dose level. After 6 participants complete their first dosing cycle, a special committee of medical professionals will meet to review the information collected on side effects, laboratory and other medical examination results to determine if it is safe to continue to the next dose level. Three dose level groups are planned for this research study, but additional dose levels may be recommended by the medical committee.

Once the optimal dose level is established, the corresponding group will be expanded, which means that additional participants will be enrolled at this dose level to enable better evaluation of the study drug safety, effects on B cells and other parameters.

Approximately 30 to 60 minutes before the infusion the subjects will receive medications that will help prevent potential side effects of the study drug administration (called premedications). These medications will help reduce inflammation, allergic reactions, and provide fever and paid relief that may occur during or shortly after the study drug infusion.

Between 12 and 36 weeks following the receipt of the initial study drug dosing cycle you may receive the additional dosing cycle, if the subject meets specific criteria, and if the study doctor considers it safe and beneficial. Additional doses will be provided at the dosing level that is approved by the medical committee at that time, which may be higher than the dose level you have received during the initial dosing cycle. Dosing visits will be conducted instead of the follow-up visits if they overlap with the follow-up from the initial dosing cycle. The following study procedures will be performed at the additional dosing visits:
Vital signs and weight
Symptom-based physical exam
Safety laboratory tests
B cell tests
Platelet tests
Antibody tests
Urinalysis
Pregnancy test (if you are a female able to get pregnant)
Study drug dosing
Measurements of the study drug concentration (dosing day only, before and at the end of infusion)
Side effect and medication check
Quality of life questionnaires (dosing Day 1 only)

### Long-Term Follow-Up Study Part:

After completing the main part of the study, the study doctor will check your B cells and advise if you should continue into the long-term follow-up part of the study. In this study part, you will come to the study site every 4 weeks for safety checks and to measure the subject's B cells. These visits will last for approximately 1-2 hours. Once the B cells return to a certain level the follow-up will be complete. This may take from several weeks to up to 52 weeks, so the overall study participation will be about 2 years. If the subject's B cells recovered while the subject was still in the main part of the study, the subject will not need to continue into the long-term follow-up part.

The following study procedures will be performed at the long-term follow-up visits:
Side effect and medication check
Vital signs and weight
Symptom-based physical exam
Complete physical exam will be done at your last long-term follow-up visit
Safety laboratory tests
B cell tests
Platelet tests
Antibody tests
Urinalysis
Pregnancy test (if you are a female able to get pregnant)
Measurements of the study drug concentration
Quality of life questionnaires
Infectious disease checks (tests for measles, mumps, rubella, tetanus, pneumococcus, and COVID-19 infections) will be done at your last long-term follow-up visit.

### Study Objectives:

### Primary Objective:

The primary objective of this study is to evaluate the safety and tolerability of ascending doses of budoprutug in subjects with ITP.

### Secondary Objectives:

The secondary objectives of this study include the following:
To investigate potential doses for subsequent dose-finding studies in subjects with ITP.
To characterize the pharmacokinetic (PK) profile of budoprutug in subjects with or ITP.
To evaluate the effects of budoprutug on B-cell depletion (pharmacodynamic [PD] response).
To evaluate the effects of budoprutug on platelet counts (ITP clinical response).

### Exploratory Objectives:

The exploratory objectives of this study include the following:
To evaluate the effects of budoprutug on serum immunoglobulin (Ig) G, IgM, and IgA levels.
To evaluate the development of anti-drug antibodies (ADAs) to budoprutug.
To evaluate subject reported outcomes/quality of life (QoL) measures.
To evaluate the PK and PK/PD (dose relationship) profile in subjects with ITP.
To evaluate the re-population of B-cells by subset immunophenotyping.
To evaluate the effects of budoprutug on anti-platelet autoantibodies.
To evaluate the changes from Baseline in antibody titers to measles, mumps, rubella, tetanus, pneumococcus, and severe acute respiratory syndrome coronavirus 2 (SARS-COV-2) in response to budoprutug.

### Study Endpoints:

The primary safety endpoints include the following:
Incidence, relatedness, severity, and duration of treatment-emergent adverse events (TEAEs) and dose-limiting toxicities (DLTs).

The secondary endpoints include the following:
Budoprutug PK parameters (including area under the maximum observed concentration, time to maximum observed concentration, terminal half-life, apparent clearance, and volume of distribution).
The change from baseline in absolute peripheral cluster of differentiation (CD) 20+ B-cell count.
The change in platelet count observed with budoprutug over time in subjects with ITP.
The percentage of subjects with ITP who achieve a stable, partial, and complete response by Week 12. Note: A stable, partial, and complete response is defined as a platelet count 30,000/μL, 50,000/μL, or 100,000/μL, respectively, on at least 2 occasions at least 7 days apart within a 30-day time frame.
The change in serum IgG, IgM, and IgA from baseline over time.
The incidence of subjects who develop ADAs at any time after study drug administration.
The percentage of subjects on a steroid at baseline who are able to stop steroid treatment.

The exploratory efficacy parameters include the following:
The change in QoL, measured using the following:
   Functional Assessment of Chronic Illness Therapy-Fatigue instrument (13-item FACIT Fatigue scale); and
   Functional Assessment of Cancer Therapy-Thrombocytopenia (6-Item FACT-Th6 scale).
Budoprutug PK and its relationship to B-cell depletion and clinical response (platelet counts).
Budoprutug dose and its relationship to B-cell depletion and clinical response (platelet counts).
The characteristics of B-cell re-population as determined by subset immunophenotyping.
Change from Baseline in levels of anti-platelet autoantibodies in response to budoprutug.
Change from Baseline in antibody titers to measles, mumps, rubella, tetanus, pneumococcus, and SARS-COV-2.

### Study Population:

This study will enroll adult male and female subjects who meet all of the inclusion criteria and none of the exclusion criteria. Subjects will be eligible for enrollment in this study regardless of race/ethnicity.

### Inclusion Criteria:

1. Willing and able to provide written informed consent.
2. Aged >18 years at the time of informed consent.
3. Platelet count <30,000/μL despite an adequate trial of at least one prior therapeutic attempt. Platelet counts of <30,000/μL must be confirmed on 2 occasions at least 5 days apart, but no more than 14 days apart.
4. Partial thromboplastin time <1.5 upper limit of normal (ULN), prothrombin time <1.5 ULN, total bilirubin <1.5 ULN unless due to Gilbert's syndrome, or an international normalized ratio <1.5 at screening.
Note: Pre-dose laboratory evaluations may be repeated one time during the screening period at the discretion of the Investigator to confirm eligibility.
5. Adequate hematologic, hepatic, and renal function, defined as the following:
a. Absolute neutrophil count (ANC)>1.5 109/L;
b. White blood cell count >3 109/L;
c. Alanine aminotransferase (ALT)<2.5 upper limit of normal (ULN);
d. Aspartate transaminase (AST)<2.5 ULN;
e. Estimated glomerular filtration rate >60 mL/min (calculated using local institutional guidelines).
   Note: Pre-dose laboratory evaluations may be repeated during the screening period at the discretion of the Investigator to confirm eligibility.
   6. If being treated with corticosteroids or thrombopoietin (TPO) agonists, subjects must be on a stable dose (<20% change in dose over the 14 days prior to the first dose of study drug). Corticosteroid treatment should not be >1 mg/kg methylprednisolone (or equivalent) for 2 weeks prior to the first dose of study drug.
   Note: Steroid pulses, short courses of steroids, or other immunosuppressive agents given as rescue therapy for clinically significant changes in platelet count may be permitted during the study after a discussion with the Medical Monitor. Steroid tapering should be considered once a clinically meaningful response in platelet count is observed.
   7. Female subjects of non-childbearing potential must be either surgically sterile (hysterectomy, bilateral tubal ligation, salpingectomy, and/or bilateral oophorectomy at least 26 weeks before the Screening Visit) or postmenopausal, defined as spontaneous amenorrhea for at least 1 year, with follicle-stimulating hormone in the postmenopausal range at Screening, as per central laboratory reference range.
   8. Female subjects of childbearing potential (i.e., ovulating, premenopausal, or not surgically sterile) must use a medically accepted, highly effective contraceptive regimen during their participation in the study and for 125 days (4 months) after the last administration of budoprutug. Highly effective contraceptive methods are defined as those with 99% or greater efficacy. Acceptable methods of contraception for female subjects of childbearing potential enrolled in the study include the following:
a. Intrauterine device for at least 12 weeks before Screening;
b. Hormonal contraception (oral, implant, injection, ring, or patch) for at least 12 weeks before Screening; or
c. True abstinence when in line with the preferred and usual lifestyle of the subject; periodic abstinence (such as calendar, ovulation, symptothermal, post-ovulation methods) and withdrawal are not acceptable methods of contraception.
   9. Male subjects must use a condom during the study and for 125 days (4 months) after their final dose of budoprutug if their partner is a female of childbearing potential. Acceptable methods of contraception for male subjects enrolled in the study include the following:
a. Condoms with spermicide; or
b. Surgical sterilization of subject at least 26 weeks before Screening (vasectomy).
   10. Male subjects must agree to abstain from sperm donation through 125 days (4 months) after the last administration of budoprutug.

### Exclusion Criteria:

1. CD19+ B-cell count <80 cells/μL at Screening or <40 cells/μL if B-cell depleting treatment was received within 24 weeks to 2 years prior to Screening.
2. Pregnant or lactating females.
3. Diagnosis of paroxysmal nocturnal hemoglobinuria, Evan's Syndrome, or any other bleeding disorder that could confound results and impact patient safety.
4. Prior treatment with rituximab or other B-cell depleting agents within 24 weeks prior to the first dose of study drug or plan to receive B-cell depleting agents during the study.
5. Current or planned treatment with any chronic anticoagulants or platelet aggregationinhibiting drugs such as aspirin, nonsteroidal anti-inflammatory drugs, or thienopyridines within 14 days of planned dosing through the end of follow-up. Symptom-based intermittent dosing of nonsteroidal anti-inflammatory drugs is permitted.
6. Prior treatment with immunosuppressants (other than corticosteroids) within 30 days or 5 times the elimination half-life (whichever is longer) of the Screening Visit (e.g., calcineurin inhibitors, mycophenolate mofetil, azathioprine), or alkylating agents within 180 days of the Screening Visit.
Note: Steroid pulses, short courses of steroids, or other immunosuppressive agents given as rescue therapy for clinically significant changes in platelet count may be permitted during the study after a discussion with the Medical Monitor.
7. Prior treatment with intravenous (IV) Ig within the last 90 days of the Screening Visit.
8. Active treatment for ITP other than steroids or TPO agonists within 30 days or 5 times the elimination half-life (whichever is longer) prior to the first dose of study drug without discussion with the Medical Monitor.
9. Treatment with any investigational drug within 30 days prior to the first dose of study drug, or at least 5 times the elimination half-life of the drug (whichever is longer), or plan to use an investigational device during the study.
10. History of malignancy within the past 5 years and likely to require chemotherapeutic or surgical treatment during the study, with the exception of non-melanoma skin cancer, cervical carcinoma in-situ, or ductal carcinoma-in-situ.
11. History of stem cell, bone marrow, or solid organ transplant.
12. Acute, chronic, or latent infection, including hepatitis B, hepatitis C or human immunodeficiency virus (HIV).
13. Evidence of active tuberculosis (TB) or being at high risk for TB based on:
a. History of active TB or untreated/incompletely treated latent TB. Subjects with a history of active or latent TB who have documentation of completion of treatment according to local guidelines and no evidence of ongoing TB may be enrolled.
b. active
c. Positive, indeterminate, or invalid interferon-gamma release assay test result at screening, unless previously treated for TB. Subjects with an indeterminate test result can repeat the test once, but if the repeat test is also indeterminate, the participant must be evaluated by a pulmonologist to determine eligibility (include with or without treatment for latent TB or exclusion).
d. Chest radiograph, chest computed tomography or magnetic resonance imaging (MRI) scan that suggests a possible diagnosis of TB or suggests that a work-up for TB should be considered; all subjects must have had lung imaging with an acceptable reading within 12 months prior to consent, or during screening.
   14. Active or uncontrolled infection at the time of informed consent or study drug initiation.
   15. Recent hospitalization for any reason within 14 days prior to Screening, unless approved by the Medical Monitor.
   16. Receipt of a live vaccine within 28 days prior to the first dose of study drug or during the study. All other vaccines must be completed within 21 days prior to the first dose of study drug.
   17. Secondary cause of ITP (e.g., malignancy, hepatitis B or C, HIV, or other autoimmune diseases [e.g., thyroiditis], or drug-induced ITP).
   18. History of drug or alcohol abuse within 5 years prior to Screening.
   19. Planned surgery during the 4 weeks following the first dose of study drug.
   20. Any other clinically significant disease, condition, or medical history that, in the opinion of the Investigator, would interfere with subject safety, study evaluations, and/or study procedures.
   21. In the opinion of the Investigator, the subject is unlikely to complete all protocol-required study visits or procedures, including follow-up visits, or comply with the study requirements for participation.

### Study Design and Duration:

This study is a Phase 1b/2a, open-label, sequential-cohort, dose escalation and expansion study to evaluate the safety, tolerability, PK, PD, and preliminary clinical effectiveness of budoprutug in subjects with ITP. The study consists of the following 2 parts:
1) Part 1-Main study
   a. Dose Escalation phase 1b
   b. Dose Expansion phase 2a
2) Part 2-Long-term safety and PD observational study

Each subject may receive additional budoprutug doses between Weeks 12 and 36 if certain safety and PD criteria are met.

Subjects will be followed through Week 48 in the main study and will subsequently enter the long-term observational part of the study for safety and B-cell repopulation follow-up. Subjects will be screened for study eligibility over 2 visits: the first visit on Days −21 to −14 (Screening) and the second visit on Days −13 to 0 (Qualifying Visit). The Qualifying Visit (Days −13 to 0) will occur at least 5 but no more than 14 days after the initial Screening Visit in order to confirm eligibility, particularly the platelet counts. After the Qualifying Visit, subjects who meet the eligibility criteria will be enrolled into the study.

### Study Duration:

The study duration for each individual subject will be as follows:

1) Main study duration will be up to 51 weeks, including the Screening and Qualifying Visits (up to 3 weeks), Treatment Period (2 weeks), and follow-up visits though Week 48.

2) Following the completion of the main study, subjects will proceed to the long-term observational part of the study and will be followed until B-cell re-population (defined as lower limit of normal or at least 80% of baseline value) which is expected to occur within 52 more weeks after the Main study.

### Dose Escalation Phase:

During the Dose Escalation phase, subjects with ITP will be assigned to the currently open cohort in the study, per the table below. Each dosing cohort will be enrolled sequentially. Six subjects will be enrolled into each dosing cohort. The DRC will review all available data for the 6 subjects included in each cohort once the last dosed subject completes their Week 6 visit (4 weeks following the second dose of study drug in the initial dosing cycle). As the subject follow-up data are generated over time, platelet counts may also be factored into the evaluation. Dosing levels will continue sequentially to determine sufficient B-cell depletion and an acceptable safety and efficacy profile for subsequent trials.

| Dose | Dose | |
|---|---|---|
| Level | (mg) | Frequency |
| Level 1 | 250 | Every 14 days for 2 doses |
| (Starting | | (500 mg cumulative dose) |
| dose) | | |
| Level 2 | 500 | Every 14 days for 2 doses |
| | | (1000 mg cumulative dose) |
| Level 3 | 1000 | Every 14 days for 2 doses |
| | | (2000 mg cumulative dose) |

In the dose escalation phase (Phase 1b), subjects will receive 2 bi-weekly doses of budoprutug at the specified dose level, administered via IV infusion. Dose escalation will be conducted using an adaptive design for each cohort, based on safety and B-cell depletion data (and any available platelet count clinical data). The first dose cohort will be 250 mg administered IV every 14 days for 2 doses (500 mg cumulative dose). The planned dose cohorts that will follow are anticipated to be 500 mg and 1000 mg administered IV every 14 days for 2 doses (1000 mg and 2000 mg cumulative dose, respectively). Each Dose Escalation cohort will be enrolled sequentially. The protocol will allow for intermediate doses or lower doses, based on the data generated in the study and recommendation of the DRC. Escalation doses between successive cohorts will not exceed more than double a previous cohort's cumulative dose. Following the first dosing cohort, the plan is flexible based on decisions of the DRC, focusing primarily on safety and PD effects of B-cell depletion. As the subject follow-up data are generated over time, platelet counts may also be factored into the evaluation.

For safety, if none of the initial 6 subjects experience a protocol-defined DLT during the initial treatment period, then enrollment into the next dose cohort can begin. If 2 of the first 6 subjects experience a DLT, then additional dosing and enrollment will be stopped. If study enrollment is stopped due to a DLT, the DRC will review all available safety data to determine if the study may resume at the current dose, resume at a lower dose, proceed to a study amendment to address safety concerns, or terminate the study. For PD effects, if the data from 6 subjects do not demonstrate consistent effects, then up to 3 additional subjects can be enrolled at that dose level to better understand the PD effects prior to escalation to the next higher dose cohort.

### Dose Expansion Phase:

The Dose Expansion phase (Phase 2a) will evaluate a cohort of subjects at the dose identified from Phase 1b. Up to 6 subjects in the expansion cohort will receive study drug at the dose identified for expansion and this cohort will also include subjects from the Dose Escalation phase treated with the expansion cohort dose.

Both the Dose Escalation and Dose Expansion phases will collect data on tolerability, PK, B-cell depletion, and complete blood counts (CBCs), including platelet counts.

### Additional Budoprutug Doses:

Following the initial dosing cycle during the first 2 weeks of the study, the same subjects may receive additional budoprutug doses, i.e., 2 additional biweekly doses of budoprutug between Weeks 12 and 36. The additional dosing is intended for study subjects who have experienced some meaningful degree of clinical response, but have not been able to maintain a complete remission, and are therefore more likely to receive additional benefit. Subjects who achieve and maintain a complete remission and those who do not respond at all are unlikely to benefit from further dosing, and therefore will not receive additional dosing. The additional dosing can be administered if criteria are met on the basis of immunologic or clinical response and the safety/tolerability profile as defined by the protocol. The additional budoprutug doses administered between Weeks 12 and 36 may be higher (or lower) than the dose received during the initial dosing cycle and will match the dose level that has been deemed safe (and potentially more effective for B-cell depletion) by the DRC. That decision would be based on available safety, PD, and other clinical data.

The additional dosing can be administered between 12 and 36 weeks if subjects satisfy the following criteria and who have not experienced any DLTs:
1. Demonstration of a clinical response to therapy at any time during the first 12 to 36 weeks of the study (defined as a platelet count >30,000/μL and a >25% platelet count increase from Baseline);
2. Circulating B-cells levels show evidence of recovery from the nadir achieved following the initial dose cycle;
3. Demonstrated loss of improvement between Weeks 12 to 36 on any of the following:
   a. Platelet count has declined by 50% from the highest count following study drug administration;
   b. Platelet count <30,000/μL;
   c. Initiation or increase in corticosteroid rescue therapy to prevent further platelet decline and continuation of corticosteroids.

The additional dosing with budoprutug should also be reviewed and approved by the medical monitor prior to administration and all activities listed in the schedule of procedures should be completed.

### On-Study Assessments Including Platelets:

Throughout the study, subjects will be seen approximately every 4 weeks to assess safety and tolerability, PD (B-cell depletion), and CBCs, including platelet count. This schedule will enable detection of changes from baseline, the time to effect, and the duration of effect. In addition, the patterns of each parameter will be evaluated. It is anticipated, if effective, that B-cell depletion will be followed by increases in platelet count in the blood.

### End of Main Study:

All subjects will have an End of Study (EOS) Visit for the main study at Week 48. This duration of follow-up allows for the collection of additional safety data and data on the persistence of B-cell depletion, the characteristics of B-cell re-population, CBCs, potential recurrence of anti-platelet antibodies, and overall ITP disease activity over time. Subjects who receive additional budoprutug doses between Weeks 12 and 36 will also be followed until Week 48 in the main study.

### Long-Term Safety and Observational Study:

Following the EOS Visit in the main study, subjects will continue to the long-term observational part of the study and will be followed until B-cell re-population, unless B-cell repopulation occurred prior to the EOS Visit in the main study. B-cell repopulation is defined as lower limit of normal or at least 80% of baseline value) which is expected to occur within 52 weeks after the Main study.

### Rescue Therapy

During the study, the Investigator may administer or prescribe rescue therapy or increase the dose of other concomitant medications (e.g., glucocorticoids or other immunosuppressive agents) for subjects with new or persistent thrombocytopenia and/or worsening symptomatology. If possible, the Medical Monitor should be consulted for advice prior to initiation.

If rescue therapy is required, the Investigator should taper the rescue medications as soon as clinically appropriate to avoid risks of over-immunosuppression and to better assess the effects of budoprutug. Subjects who receive rescue medication should continue to be monitored per study requirements as indicated in the Schedule of Procedures.

### Dosage Forms and Route of Administration:

Subjects will be required to receive premedication prior to each infusion of budoprutug to reduce the frequency and severity of infusion-related reaction (IRRs), as shown in the table below.

### Premedication Prior to Each Budoprutug Infusion:

| | | | Administration | |
|---|---|---|---|---|
| Type of | Route of | Examples | Time Prior to | |
| Medication | Administration | (Or Equivalent) | Budoprutug Infusion | |
| Corticosteroid | Intravenous | Methylprednisolone | 30 | minutes |
| | | (or steroid equivalent) | | |
| | | 0.5 mg/kg to 1 mg/kg ^{a} | | |
| Antihistamine | Oral | Diphenhydramine | 30 to 60 | minutes |
| | (preferred) | 25 mg to 50 mg | | |
| Antipyretic^{b} | Oral | Acetaminophen/paracetamol | 30 to 60 | minutes |
| | | 500 mg to 650 mg | | |

^{a} The dose may be reduced or held if the subject is already on corticosteroids.
^{b}Nonsteroidal anti-inflammatory drugs (NSAIDs) should not be used due to their potential effect on platelets.

Premedications outlined above are considered auxiliary medicinal products. Only authorized auxiliary medicinal products will be used for premedication, and their use must be in accordance with the terms of their marketing authorizations. If premedication equivalents need to be used based on local sourcing availability, they will be selected from medicinal products that have a current marketing authorization.

Budoprutug will be provided as a concentrated solution. Budoprutug will be diluted to prepare a solution of IV infusion. The infusion solution preparation and administration time must not exceed 4 hours.

During the Dose Expansion phase, the infusion rate may be the same or higher than the rate used during the Dose Escalation phase based upon DRC review of available safety data. Subjects will be observed for at least 1 hour following each infusion to monitor for potential infusion reactions.

The table below provides the sequences for nucleic acids, proteins, and peptides discussed herein.

| SEQ ID | |
|---|---|
| NO: | Sequence |
| 1 | CAGGTGCAACTGCAGCAGCCTGGGGCTGAAGTGGTGAAGCCTGGGGC |
| | TTCAGTGAGACTGTCCTGCAAGACTTCTGGCTACACCTTCACCAGCAA |
| | CTGGATGCACTGGGTGAAGCAGAGGCCTGGACAAGGCCTTGAGTGGA |
| | TCGGAGAGATTGATCCTTCTGATAGTTATACTAACTACAATCAAAAGTT |
| | CAAGGGCAAGGCCAAGTTGACTGTAGACAAATCCTCCAGCACAGCCT |
| | ACATGGAAGTCAGCAGCCTGACATCTGAGGACTCTGCGGTCTATTACT |
| | GTGCAAGAGGTAGCAACCCTTACTACTATGCTATGGACTACTGGGGTCA |
| | AGGAACCTCAGTCACCGTCTCCTCA |
| 2 | CAGGTGCAACTGCAGCAGCCTGGGGCTGAAGTGGTGAAGCCTGGGGC |
| | TTCAGTGAGACTGTCCTGCGAGACTTCTGGCTACACCTTCACCAGCAA |
| | CTGGATGCACTGGGTGAAGCAGAGGCCTGACCAAGGACTTGAGTGGA |
| | TCGGAGAGATTGATCCTTCTGATAGTTATACTAACTACAATCAAAAGTT |
| | CAAGGGCAAGGCCGAATTGACTGTAGACAAATCCTCCAGCACAGCCTA |
| | CATGGAAGTCAGCGACCTGACATCTGAGGACTCTGCGGTCTATTACTGT |
| | GCAAGAGGTAGCAACCCTTACTACTATGCTATGGACTACTGGGGTCAA |
| | GGAACCTCAGTCACCGTCTCCTCA |
| 3 | CAGGTGCAACTGCAGCAGCCTGGGGCTGAAGTGGTGAAGCCTGGGGC |
| | TTCAGTGAGACTGTCCTGCAAGACTTCTGGCTACACCTTCACCAGCAA |
| | CTGGATGCACTGGGTGAAGCAGAGACCTGGACAAGGACTTGAGTGGA |
| | TCGGAGAGATTGATCCTTCTGATAGTTATACTAACTACAATCAAAAGTT |
| | CAAGGGCAAGGCCGAATTGACTGTAGACAAATCCTCCAGCACAGCCTA |
| | CATGGAAGTCAGCGACCTGACATCTGAGGACTCTGCGGTCTATTACTGT |
| | GCAAGAGGTAGCAACCCTTACTACTATGCTATGGACTACTGGGGTCAA |
| | GGAACCTCAGTCACCGTCTCCTCA |
| 4 | CAGGTGCAACTGGAGCAGCCTGGGGCTGAAGTGAAGAAGCCTGGGGC |
| | TTCAGTGAAGGTGTCCTGCAAGGCTTCTGGCTACACCTTCACCAGCAA |
| | CTGGATGCACTGGGTGAAGCAGAGGCCTGGACAAGGACTTGAGTGGA |
| | TCGGAGAGATTGATCCTTCTGATAGTTATACTAACTACAATCAAAAGTT |
| | CAAGGGCAAGGCCAAGTTGACTGTAGACAAATCCTCCAGCACAGCCT |
| | ACATGGAAGTCAGCGACCTGACAGCTGAGGACTCTGCGGTCTATTACT |
| | GTGCAAGAGGTAGCAACCCTTACTACTATGCTATGGACTACTGGGGTCA |
| | AGGAACCTCAGTCACCGTCTCCTCA |
| 5 | CAGGTGCAACTGGAGCAGCCTGGGGCTGAAGTGGTGAAGCCTGGGGC |
| | TTCAGTGAAGGTGTCCTGCAAGACTTCTGGCTACACCTTCACCAGCAA |
| | CTGGATGCACTGGGTGAAGCAGACGCCTGGAAAAGGACTTGAGTGGA |
| | TCGGAGAGATTGATCCTTCTGATAGTTATACTAACTACAATCAAAAGTT |
| | CGATGGCAAGGCCAAGTTGACTGTAGACAAATCCTCCAGCACAGCCTA |
| | CATGGAAGTCAGCGACCTGACAGCTGAGGACTCTGCGACCTATTACTG |
| | TGCAAGAGGTAGCAACCCTTACTACTATGCTATGGACTACTGGGGTCAA |
| | GGAACCTCAGTCACCGTCTCCTCA |
| 6 | CAGGTGCAACTGGAGCAGCCTGGGGCTGAAGTGAAGAAGCCTGGGGC |
| | TTCAGTGAAGGTGTCCTGCAAGGCTTCTGGCTACACCTTCACCAGCAA |
| | CTGGATGCACTGGGTGAGACAGGCACCTGGAAAAGGACTTGAGTGGA |
| | TCGGAGAGATTGATCCTTCTGATAGTTATACTAACTACAATCAAAAGTT |
| | CGATGGCAAGGCCAAGTTGACTGTAGACAAATCCTCCAGCACAGCCTA |
| | CATGGAAGTCAGCGACCTGACATCTGAGGACTCTGCGACCTATTACTG |
| | TGCAAGAGGTAGCAACCCTTACTACTATGCTATGGACTACTGGGGTCAA |
| | GGAACCTCAGTCACCGTCTCCTCA |
| 7 | CAGGTGCAACTGGAGCAGCCTGGGGCTGAAGTGGTGAAGCCTGGGGC |
| | TTCAGTGAAGGTGTCCTGCAAGACTTCTGGCTACACCTTCACCAGCAA |
| | CTGGATGCACTGGGTGAAGCAGAGGCCTGGACAAGGACTTGAGTGGA |
| | TCGGAGAGATTGATCCTTCTGATAGTTATACTAACTACAATCAAAAGTT |
| | CGATGGCAAGGCCAAGTTGACTGTAGACAAATCCTCCAGCACAGCCTA |
| | CATGGAAGTCAGCGACCTGACATCTGAGGACTCTGCGACCTATTACTG |
| | TGCAAGAGGTAGCAACCCTTACTACTATGCTATGGACTACTGGGGTCAA |
| | GGAACCTCAGTCACCGTCTCCTCA |
| 8 | CAAATTGTTCTCACCCAGTCTCCAGCAATCATGTCTGCATCTCCAGGGG |
| | AGAAGGTCACCATGACCTGCAGTGCCAGCTCAGGTGTCAACTACATGC |
| | ACTGGTACCAGCAGAAGCCAGGCACCTCCCCCAAAAGATGGATTTATG |
| | ACACATCCAAACTGGCTTCTGGAGTCCCTGCTCGCTTCAGTGGCAGTG |
| | GGTCTGGGACCTCTTATTCTCTCACAATCAGCAGCATGGAGGCTGAAG |
| | ATGCTGCCACTTATTACTGCCATCAGCGAGGTAGTTACACGTTCGGAGG |
| | GGGGACCAAGCTGGAAATAAA |
| 9 | CAAATTGCTCTCACCCAGGAGCCAGCAATCATGTCTGCATCTCCAGGG |
| | GAGAAGGTCACCATGACCTGCAGTGCCAGCTCAGGTGTCAACTACATG |
| | CACTGGTATCAGCAGAAGCCAGGCACCTCCCCCAAAAGATGGATTTAT |
| | GACACATCCAAACTGGATTCTGGAGTCCCTGCTCGCTTCAGTGGCAGT |
| | GGGTCTGGGACCTCTTATTCTCTCACAATCAGCAGCATGGAGGCTGAA |
| | GATGCTGCCACTTATTACTGCCATCAGCGAGGTAGTTACACGTTCGGAG |
| | GGGGGACCAAGCTGGAAATAAA |
| 10 | GACATTGTTCTCACCCAGTCTCCAGCAACTTTGTCTGCATCTCCAGGGG |
| | AGAAGGTCACCATGACCTGTAGTGCCAGCTCAGGTGTCAACTACATGC |
| | ACTGGTATCAGCAGAAGCCAGGCACCTCCCCCAAAAGATGGATTTATG |
| | ACACATCCAAACTGGATTCTGGAGTCCCTGCTCGCTTCAGTGGCAGTG |
| | GGTCTGGGACCTCTTATTCTCTCACAATCAGCAGCATGGAGGCTGAAG |
| | ATGCTGCCACTTATTACTGCCATCAGCGAGGTAGTTACACGTTCGGAGG |
| | GGGGACCAAGCTGGAAATAAA |
| 11 | CAAATTGTTCTCACCCAGTCTCCAGCAACTTTGTCTGCATCTCCAGGGG |
| | AGAAGGCTACCATGACCTGCAGTGCCAGCTCAGGTGCTAACTACATGC |
| | ACTGGTACCAGCAGAAGCCAGGCACCTCCCCCAAAAGATGGATTTATG |
| | ACACATCCAAACTGGCTTCTGGAGTCCCTGCTCGCTTCAGTGGCAGTG |
| | GGTCTGGGACCTCTTATTCTCTCACAATCGAGAGCATGGAGGCTGAAG |
| | ATGCTGCCACTTATTACTGCCATCAGCGAGGTAGTTACACGTTCGGAGG |
| | GGGGACCAAGCTGGAAATAAA |
| 12 | CAAATTGTTCTCACCCAGTCTCCAGCAACTTTGTCTGCATCTCCAGGGG |
| | AGAAGGCTACATGACCTGTAGTGCCAGCTCAGGTGTCAACTACATGCA |
| | CTGGTACCAGCAGAAGCCAGGCACCTCCCCCAAAAGATGGATTTATGA |
| | CACAGACAAAACGGCTTCTGGAGTCCCTGCTCGCTTCAGTGGCAGTGG |
| | GTCTGGGACCTCTTATTCTCTCACAATCAGCAGCATGGAGGCTGAAGAT |
| | GCTGCCACTTATTACTGCCATCAGCGAGGTAGTTACACGTTCGGAGGG |
| | GGGACCAAGCTGGAAATAAA |
| 13 | QVQLQQPGAEVVKPGASVRLSCKTSGYTFTSNWMHWVKQRPGQGLEWI |
| | GEIDPSDSYTNYNQKFKGKAKLTVDKSSSTAYMEVSSLTSEDSAVYYCAR |
| | GSNPYYYAMDYWGQGTSVTVSS |
| 14 | QVQLQQPGAEVVKPGASVRLSCETSGYTFTSNWMHWVKQRPDQGLEWI |
| | GEIDPSDSYTNYNQKFKGKAELTVDKSSSTAYMEVSDLTSEDSAVYYCAR |
| | GSNPYYYAMDYWGQGTSVTVSS |
| 15 | QVQLQQPGAEVVKPGASVRLSCKTSGYTFTSNWMHWVKQRPGQGLEWI |
| | GEIDPSDSYTNYNQKFKGKAELTVDKSSSTAYMEVSDLTSEDSAVYYCAR |
| | GSNPYYYAMDYWGQGTSVTVSS |
| 16 | QVQLEQPGAEVKKPGASVKVSCKASGYTFTSNWMHWVKQRPGQGLEW |
| | IGEIDPSDSYTNYNQKFKGKAKLTVDKSSSTAYMEVSDLTAEDSAVYYCA |
| | RGSNPYYYAMDYWGQGTSVTVSS |
| 17 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSS |
| 18 | QVQLEQPGAEVKKPGASVKVSCKASGYTFTSNWMHWVRQAPGKGLEW |
| | IGEIDPSDSYTNYNQKFDGKAKLTVDKSSSTAYMEVSDLTSEDSATYYCA |
| | RGSNPYYYAMDYWGQGTSVTVSS |
| 19 | QVQLEQPGAEVVKPGASVKVSCKTSGYTFTSNWMHWVKQRPGQGLEWI |
| | GEIDPSDSYTNYNQKFDGKAKLTVDKSSSTAYMEVSDLTSEDSATYYCAR |
| | GSNPYYYAMDYWGQGTSVTVSS |
| 20 | QVQLQQPGAEVKKPGASVRLSCETSGYTFTSNWMHWVKQRPDQGLEWI |
| | GEIDPSDSYTNYNQKFDGKAELTVDKSSSTAYMEVSDLTSEDSAVYYCAR |
| | GSNPYYYAMDYWGQGTSVTVSS |
| 21 | QVQLEQPGAEVKKPGASVKVSCKASGYTFTSNWMHWVKQTPGKGLEW |
| | IGEIDPSDSYTNYNQKFDGKAKLTVDKSSSTAYMEVSDLTAEDSATYYCA |
| | RGSNPYYYAMDYWGQGTSVTVSS |
| 22 | QVQLXQPGAEVXKPGASVXXSCXXSGYTF |
| 23 | WVXQXPXXGLEWIG |
| 24 | YNQKFXGKAXLTVDKSSSTAYMEVSXLTXEDSAXYYCA |
| 25 | QIVLTQSPAIMSASPGEKVTMTCSASSGVNYMHWYQQKPGTSPKRWIYD |
| | TSKLASGVPARFSGSGSGTSYSLTISSMEAEDAATYYCHQRGSYTFGGGT |
| | KLEIK |
| 26 | QIALTQEPAIMSASPGEKVTMTCSASSGVNYMHWYQQKPGTSPKRWIYD |
| | TSKLDSGVPARFSGSGSGTSYSLTISSMEAEDAATYYCHQRGSYTFGGGT |
| | KLEIK |
| 27 | DIVLTQSPATLSASPGEKVTMTCSASSGVNYMHWYQQKPGTSPKRWIYDT |
| | SKLDSGVPARFSGSGSGTSYSLTISSMEAEDAATYYCHQRGSYTFGGGTK |
| | LEIK |
| 28 | QIVLTQSPATLSASPGEKATMTCSASSGANYMHWYQQKPGTSPKRWIYDT |
| | SKLASGVPARFSGSGSGTSYSLTIESMEAEDAATYYCHQRGSYTFGGGTK |
| | LEIK |
| 29 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIK |
| 30 | QIALTQEPAIMSASPGEKATMTCSASSGVNYMHWYQQKPGTSPKRWIYD |
| | TSKLDSGVPARFSGSGSGTSYSLTIESMEAEDAATYYCHQRGSYTFGGGT |
| | KLEIK |
| 31 | QIVLTQSPATLSASPGEKATMTCSASSGANYMHWYQQKPGTSPKRWIYDT |
| | DKTASGVPARFSGSGSGTSYSLTIESMEAEDAATYYCHQRGSYTFGGGTK |
| | LEIK |
| 32 | XIXLTQXPAXXSASPGEKXTMTC |
| 33 | DTXKXX |
| 34 | QVQLQQPGAEVVKPGASVRLSCKTSGYTFTSNWMHWVKQRPGQGLEWI |
| | GEIDPSDSYTNYNQKFKGKAKLTVDKSSSTAYMEVSSLTSEDSAVYYCAR |
| | GSNPYYYAMDYWGQGTSVTVSS |
| 35 | QIVLTQSPAIMSASPGEKVTMTCSASSGVNYMHWYQQKPGTSPKRWIYD |
| | TSKLASGVPARFSGSGSGTSYSLTISSMEAEDAATYYCHQRGSYTFGGGT |
| | KLEIK |
| 36 | VRLSCKTSG |
| 37 | WVKQRPGQG |
| 38 | YNQKFKGKA |
| 39 | FKGKAKLTV |
| 40 | YMEVSSLTS |
| 41 | VYYCARGSN |
| 42 | IVLTQSPAI |
| 43 | VLTQSPAIM |
| 44 | VTMTCSASS |
| 45 | VNYMHWYQQ |
| 46 | WIYDTSKLA |
| 47 | IYDTSKLAS |
| 48 | CGTAAGTGGATCC |
| 49 | GCTAGCTCCAGC |
| 50 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLS |
| | PGK |
| 51 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLS |
| | PGK |
| 52 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |
| 53 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |
| 54 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLS |
| | PGK |
| 55 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of treating a subject having immune thrombocytopenia (ITP) comprising administering to the subject a composition comprising a therapeutically effective amount an anti-CD19 antibody, wherein the anti-CD19 antibody comprises: a heavy chain variable region at least 95% identical to SEQ ID NO: 17 and/or a light chain variable region at least 95% identical to SEQ ID NO: 29.

2. The method of claim 1, wherein the anti-CD19 antibody comprises a heavy chain variable region comprising SEQ ID NO: 17 and/or a light chain variable region comprising SEQ ID NO: 29, and preferably the anti-CD19 antibody is budoprutug.

3. The method of claim 2, wherein the composition is administered parenterally.

4. The method of claim 3, wherein the composition is administered via an intravenous (IV) infusion.

5. The method of claim 4, wherein the composition is administered at days 1 and 15 at the start of the treatment.

6. The method of claim 5, wherein the composition is further administered at days 169 and 183 after the start of the treatment.

7. The method of claim 6, wherein the composition is administered in treatment cycles, each treatment cycle comprising administration of the composition at days 1 and 15 at start of each treatment cycle, wherein there is a 12 to 36-week interval between start of each said treatment cycle.

8. The method of claim 7, wherein said interval between each treatment cycle is 12 weeks.

9. The method of claim 7, wherein said interval between each treatment cycle is 24 weeks.

10. The method of claim 7, wherein said interval between each treatment cycle is 36 weeks.

11. The method of claim 7, wherein the composition comprises about 100 mg to about 1000 mg of the anti-CD19 antibody.

12. (canceled)

13. (canceled)

14. (canceled)

15. The method of claim 11, wherein the composition comprises about 200 mg of the anti-CD19 antibody.

16. The method of claim 11, wherein the composition comprises about 600 mg of the anti-CD19 antibody.

17. The method of claim 11, wherein the composition comprises about 1000 mg of the anti-CD19 antibody.

18. The method of claim 11, wherein the method further comprises:
measuring concentration for markers of ITP progression in the subject; and
optionally administering an additional dosing cycle of the composition to the subject.

19. The method of claim 18, wherein the markers are selected from the group consisting of platelet count and B cells.

20. The method of claim 19, wherein the additional dosing cycle is administered if the markers of ITP progression demonstrate insufficient response after administration of initial dose of the composition.

21. The method of claim 18, wherein the measuring step is conducted about 8 to about 16 weeks after administration of initial dose of the compositions.

22. The method of claim 18, wherein the measuring step is conducted about at least 12 weeks after administration of initial dose of the compositions.

23. The method of claim 18, wherein the measuring step is conducted about 12 weeks after administration of initial dose of the compositions.

24.-57. (canceled)
